(19) United States
(12) Patent Application Publication
(10) Pub. No.: US 2008/0039487 A1
(43) Pub. Date: Feb. 14, 2008

(54) Processes and intermediates useful for preparing integrase inhibitor compounds

(75) Inventors: Jared W. Evans, Foster City, CA (US); Jay Parrish, Redwood City, CA (US); Dominika Pcion, Half Moon Bay, CA (US); Richard Polniaszek, Redwood City, CA (US); Christina J. Schmidt, Belmont, CA (US); Richard H. Yu, San Francisco, CA (US); Vahid Zia, San Carlos, CA (US)

Correspondence Address: GILEAD SCIENCES INC, 333 LAKESIDE DR, FOSTER CITY, CA, 94404, US

(73) Assignee: GILEAD SCIENCES, LLC

(21) Appl. No.: 11/644,811

(22) Filed: Dec. 21, 2006

Related U.S. Application Data
(60) Provisional application No. 60/752,823, filed on Dec. 21, 2005.

(51) Int. Cl. A61K 31/44; A61P 31/18; C07D 471/04

(52) U.S. Cl. 514/292; 546/84

(57) ABSTRACT

The invention provides processes and intermediates useful for preparing integrase inhibiting compounds.

### PRIORITY OF INVENTION

This application claims priority to U.S. Provisional Application No. 60/752,823, filed on 21 Dec. 2005.

### BACKGROUND OF THE INVENTION

Human immunodeficiency virus (HIV) infection and related diseases are a major public health problem worldwide. A virally encoded integrase protein mediates specific incorporation and integration of viral DNA into the host genome. Integration is necessary for viral replication. Accordingly, inhibition of HIV integrase is an important therapeutic pursuit for treatment of HIV infection of the related diseases.

Human immunodeficiency virus type 1 (HIV-1) encodes three enzymes which are required for viral replication: reverse transcriptase, protease, and integrase. Although drugs targeting reverse transcriptase and protease are in wide use and have shown effectiveness, particularly when employed in combination, toxicity and development of resistant strains have limited their usefulness (Palella, etal N. Engl. J. Med. (1998) 338:853-860; Richman, D. D. Nature (2001) 410:995-1001). There is a need for new agents directed against alternate sites in the viral life cycle. Integrase has emerged as an attractive target, because it is necessary for stable infection and homologous enzymes are lacking in the human host (LaFemina, etal J. Virol. (1992) 66:7414-7419). The function of integrase is to catalyze integration of proviral DNA, resulting from the reverse transcription of viral RNA, into the host genome, by a stepwise fashion of endonucleolytic processing of proviral DNA within a cytoplasmic preintegration complex (termed 3′-processing or “3′-P”) with specific DNA sequences at the end of the HIV-1 long terminal repeat (LTR) regions, followed by translocation of the complex into the nuclear compartment where integration of 3′-processed proviral DNA into host DNA occurs in a “strand transfer” (ST) reaction (Hazuda, etal Science (2000) 287:646-650; Katzman, etal Adv. Virus Res. (1999) 52:371-395; Asante-Applah, etal Adv. Virus Res. (1999) 52:351-369). Although numerous agents potently inhibit 3′-P and ST in extracellular assays that employ recombinant integrase and viral long-terminal-repeat oligonucleotide sequences, often such inhibitors lack inhibitory potency when assayed using fully assembled preintegration complexes or fail to show antiviral effects against HIV-infected cells (Pommier, etal Adv. Virus Res. (1999) 52:427-458; Farnet, etal Proc. Natl. Acad. Sci. U.S.A. (1996) 93:9742-9747; Pommier, etal Antiviral Res. (2000) 47:139-148. International Patent Application Publication Number WO 2006/125048 describes certain compounds that are reported to be useful as HIV integrase inhibitors and as anti-HIV agents. One particular compound described therein is a compound of Formula (II),

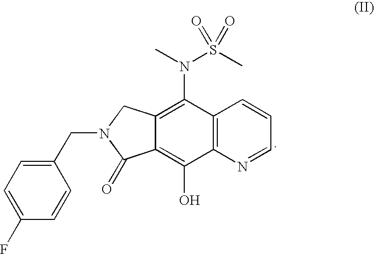

Currently there is a need for improved synthetic methods and synthetic intermediates that can be used to prepare a compound of Formula (II) or a salt thereof.

There is also a need for salts of a compound of Formula (II) that possess useful therapeutic and/or physical properties (e.g. improved solubility or oral bioavailability), or physical properties that improve the ability of the compound to be formulated for administration as a therapeutic agent.

### SUMMARY OF THE INVENTION

In one embodiment the invention provides a method of preparing a compound of Formula (I),

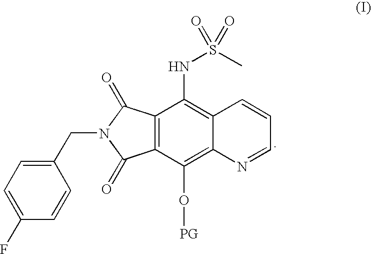

In one embodiment the invention provides a method of preparing a compound of Formula (II),

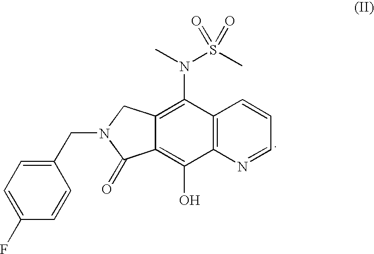

In one embodiment the invention provides salts of a compound of formula (II) described herein.

In one embodiment the invention provides novel synthetic intermediates and processes described herein that are useful for preparing an integrase inhibitor of formula (II); or a salt thereof.

### DETAILED DESCRIPTION OF THE INVENTION

As used herein, N-(7-(4-fluorobenzyl)-9-hydroxy-8-oxo-7,8-dihydro-6H-pyrrolo[3,4-g]quinolin-5-yl)-N-methylmethanesulfonamide is also referred to as a compound of the Formula (II),

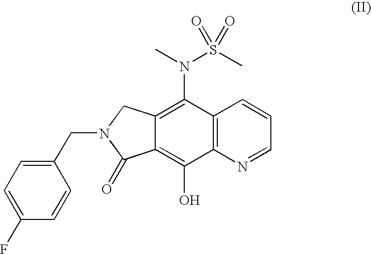

As used herein the term “protecting group” refers to a moiety of a compound that masks or alters the properties of a functional group or the properties of the compound as a whole. Chemical protecting groups and strategies for protection/deprotection are well known in the art. See e.g., Protective Groups in Organic Chemistry, Third Edition Theodora W. Greene and Peter G. M. Wuts, John Wiley & Sons, Inc., New York, 1999. Protecting groups are often utilized to mask the reactivity of certain functional groups, to assist in the efficiency of desired chemical reactions, e.g., making and breaking chemical bonds in an ordered and planned fashion. Protection of functional groups of a compound alters other physical properties besides the reactivity of the protected functional group, such as the polarity, lipophilicity (hydrophobicity), and other properties which can be measured by common analytical tools. Chemically protected intermediates may themselves be biologically active or inactive. Examples of types of protecting groups are silyl-ethers and carbon based ethers such as benzyl ethers. Variously substituted alkyl and aryl substituted silyl-ethers are known to be useful protecting groups, these include but are not limited trimethylsilyl (TMS) ether, t-butyldimethylsilyl (TBDMS) ether, t-butyldiphenylsilyl (TBDPS) ether and triisopropylsilyl (TIPS) ether.

Additional protecting groups include but are not limited to: alkyl ethers such as methyl, cyclopropylmethyl, allyl, isopropyl, cyclohexyl, t-butyl, benzyl, 2,6-dimethylbenzyl, 4-methoxybenzyl, diarylmethyl, o-nitrobenzyl, 2,6-dichlorobenzyl, 4-(dimethylaminocarbonyl)benzyl, 9-anthrylmethyl, or 4-picolyl; aryl ethers such as heptafluoro-p-tolyl or tetrafluoro-4-pyridyl; alkoxyalkyl such as methoxymethyl (MOM), benzyloxymethyl (BOM), methoxyethoxymethyl (MEM), 2-(trimethylsilyl)ethoxymethyl (SEM), methylthiomethyl (MTM), phenylthiomethyl (PTM), 2,2-dichloro-1,1-difluoroethyl, tetrahydrophyranyl (THP), phenacyl, or p-bromophenacyl; esters such as aryl acetate, aryl levulinate, aryl pivaloate, aryl benzoate, or aryl 9-fluorenecarboxylate; carbonates such as aryl methyl carbonate, aryl 2,2,2-trichloroethyl, aryl vinyl, aryl benzyl, or aryl carbamates; phosphinates such as dimethylphosphinyl (Dmp) or dimethylthiophosphinyl (Mpt); sulfonates such as aryl methanesulfonate, aryl toluenesulfonate, or aryl 2-formylbenzenesulfonate.

As used herein, a “protecting agent” refers to a reagent which effects the addition of a protecting group (e.g. the selective addition ) to a compound.

As used herein, a “deprotection agent” refers to a reagent which effects the removal of a protecting group (e.g. the selective removal) from a compound. The typical deprotection agents will vary by the protecting group they are intended to remove and are well known in the art. For example, trialkyl silyl groups can be removed by acids, such as trifluoroacetic acid, or by tetralkylammonium fluorides, as well as other reagents described in texts such as Protective Groups in Organic Chemistry, Third Edition, Theodora W. Greene and Peter G. M. Wuts, John Wiley & Sons, Inc., New York, 1999. The choice of “deprotecting agent” will depend not only on the “protecting group” to be removed, but also on the nature of the compound the protecting group is being removed from. For example, to remove a silyl group from a compound which is otherwise unstable to acid, a reagent capable of supplying a fluoride anion can be used. To remove a silyl group from a molecule containing alkali sensitive functionality, an agent such as trifluoroacetic acid may be used.

As used herein, the term “reducing agent” refers to reagents used for the reduction of a carbonyl functionality to the corresponding alcohol, amine, or aminal. Examples of reducing agents/systems and methods include, but are not limited to: Borohydrides such as NaBH₄, NaBH₄ and a additive (such as TiCl₄, CoCl₂.6 H₂O, AlCl₃, HOAc, MeSO₃H, pyridine, CF₃CH₂OH, (Et₃O)BF₄, SnCl₄, POCl₃, HSCH₂CH₂SH), KBH₄; P₂S₅/MeI/NaBH₄ or NaBH₃CN, (p-MeOC₆H₄)₂P₂S₄/(Et₃O)BF₄/NaBH₄, LiBH₃CN, LiBH₄/MeOH/diglyme, or dimethylaminoborohydride; boranes such as BH₃, BH₃.SMe₂, BH₃.SMe₂/BF₃.Et₂O, or catechol borane; aluminum hydrides such as AlH₃, i-BU₂AlH, LiAlH₄, NaH₂Al(OCH₂C₂OCH₃)₂ or LiHAl(OCH₃)₃; other methods and systems such as Mg, Zn dust/acetic acid, P₄S₁₀/Raney nickel, sodium/n-propanol or trichlorosilane. Additional methods include: hydrogenation such as Pd/H₂/TFA; photochemical reactions or ultrasonic reactions. As used herein, the term “methylation reagent” to a reagent that can be used for covalent attaching a methyl group to a nitrogen atom. Examples of methylation reagents include but are not limited to: dimethylsulfate, methyl iodide, methyl chloride, and methyl bromide.

Isolation and purification of the compounds and intermediates described herein can be effected, if desired by any suitable separation or purification procedure such as for example, filtration, extraction, crystallization, column chromatography, thin-layer chromatography or thick-layer chromatography, or a combination of these procedures. Specific illustrations of suitable separation and isolation procedures are disclosed in the examples herein. Other equivalent separation and isolation procedures known to those of ordinary skill in the art may also be utilized.

As used herein, the following abbreviations refer to the corresponding terms.

AN Area Normalization

DMAP 4-(dimethylamino)pyridine

ES External Standard

NaHMDS sodium hexamethyldisilazane

rt room temperature

TIPS triisopropylsilyl

KF Karl Fischer titration

Specific Embodiments of the Invention Specific embodiments of the invention described herein are for illustration; they do not exclude other aspects of the invention described herein.

A specific embodiment of the invention provides a compound of Formula (I) 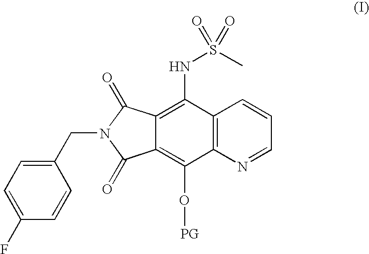
wherein PG is a protecting group useful in a process for preparing a compound of Formula (II)

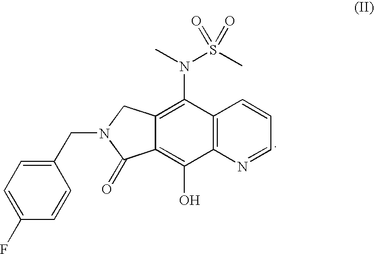

A specific embodiment of the invention provides a method of preparing a compound or a pharmaceutically acceptable salt of Formula (II) 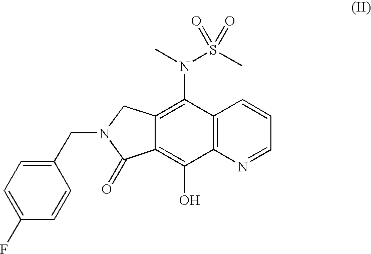
comprising contacting a compound of the Formula (III), 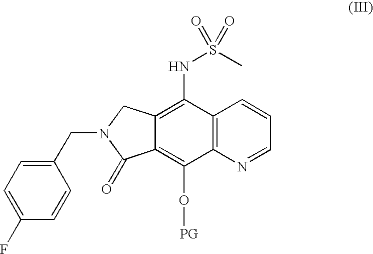
wherein PG is a protecting group, with a methylation agent and an acid.

In one specific embodiment of the invention treatment of the methylation agent is followed by an acid.

In one specific embodiment of the invention the protecting group is a silyl-ether.

In one specific embodiment of the invention the silyl-ether is an alkyl substituted silyl-ether.

In one specific embodiment of the invention the alkyl substituted silyl ether is tri-isopropylsilyl ether.

In one specific embodiment of the invention the methylation reagent is a methyl halide.

In one specific embodiment of the invention the methyl halide is methyl iodide.

In one specific embodiment of the invention the methylation reagent is methyl iodide or dimethylsulfate.

In one embodiment of the invention a compound of Formula (I), 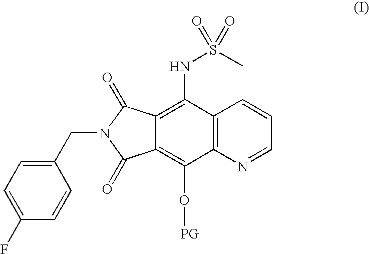
is contacted with a reducing agent to provide the compound of the Formula (III), 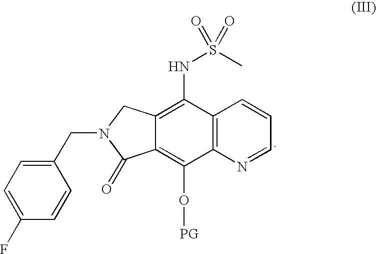
Reducing a compound of Formula (I) to a compound of Formula (III) provides an improved synthetic pathway for preparing a compound of Formula (II), since it eliminates side reactions and additional steps from the processes described in International Patent Application Publication Number WO 2006/125048.

In one specific embodiment of the invention the reducing reagent is a borohydride.

In one specific embodiment of the invention the borohydride is LiBH₄.

One specific embodiment of the invention provides a method of preparing a compound of Formula (II) 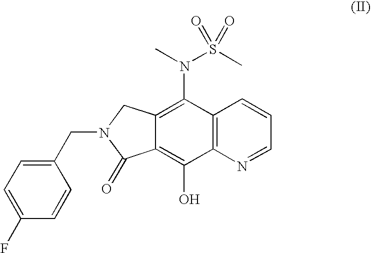
comprising contacting a compound of the Formula,

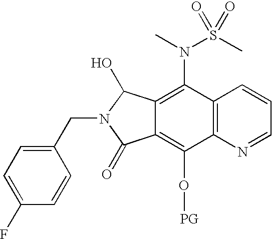

wherein, PG is a protecting group, with a reducing agent and an acid.

In one specific embodiment of the invention the protecting group is a silyl-ether.

In one specific embodiment of the invention the silyl-ether is an alkyl substituted silyl-ether.

In one specific embodiment of the invention the alkyl substituted silyl ether is tri-isopropylsilyl ether.

In one specific embodiment of the invention the reducing reagent is a borohydride.

In one specific embodiment of the invention the borohydride is LiBH₄.

In one specific embodiment the invention further comprises contacting a compound of the Formula, 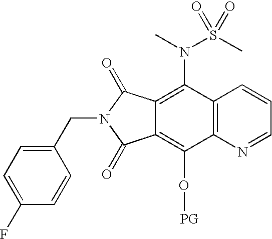
with a reducing agent to provide the compound of the Formula,

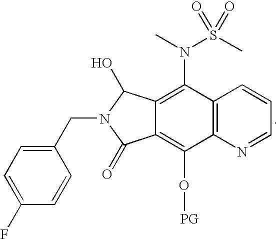

In one specific embodiment of the invention the reducing reagent is a borohydride.

In one specific embodiment of the invention the borohydride is LiBH₄.

In one specific embodiment the invention further comprises contacting a compound of the Formula (I): 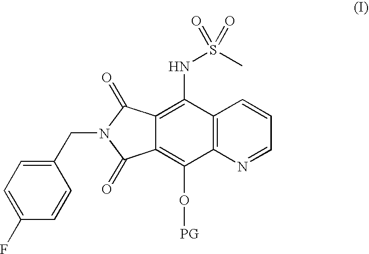
with a methylation reagent to provide the compound of the Formula

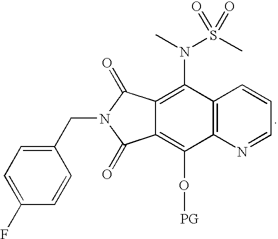

In one specific embodiment of the invention the methylation reagent is a methyl halide.

In one specific embodiment of the invention the methyl halide is methyl iodide.

In one specific embodiment of the invention the methylation reagent is methyl iodide or dimethylsulfate.

In one specific embodiment the invention provides a method of preparing a compound of Formula (I) 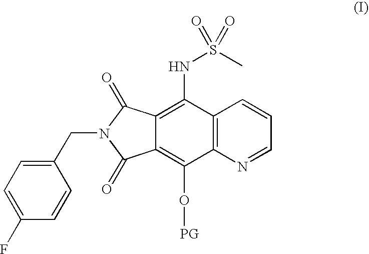
comprising contacting a compound of Formula 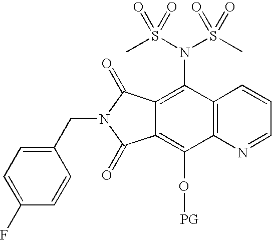
with a base.

In one specific embodiment of the invention the base is a strong base.

In one specific embodiment of the invention the strong base is potassium hydroxide.

In one specific embodiment the invention further comprises contacting a compound of Formula 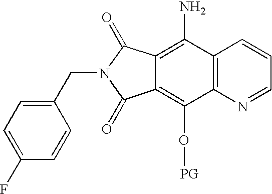
with a base and a methyl sulfonation reagent to provide a compound of the Formula

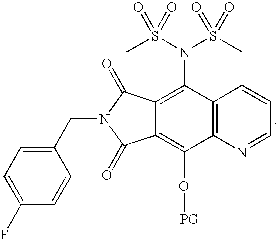

In one specific embodiment of the invention the methyl sulfonation reagent is methylsulfonyl chloride.

In one specific embodiment the invention further comprises contacting a compound of Formula 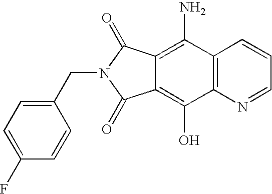
with a protecting agent to provide a compound of the Formula

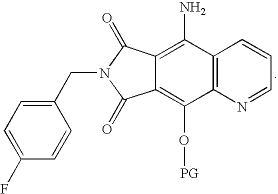

In one specific embodiment of the invention the protecting group is a silyl-ether.

In one specific embodiment of the invention the silyl-ether is an alkyl substituted silyl-ether.

In one specific embodiment of the invention the alkyl substituted silyl ether is tri-isopropylsilyl ether.

In one specific embodiment the invention further comprises contacting a compound of Formula 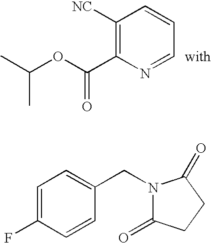
to provide a compound of the Formula

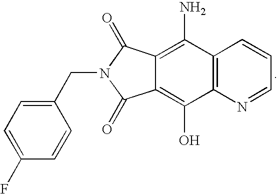

In one specific embodiment the invention further comprises contacting a compound of the Formula 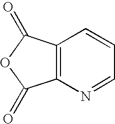
with isopropanol to provide a compound of the Formula

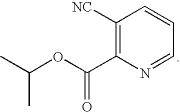

In one specific embodiment the invention further comprises contacting a compound of the Formula 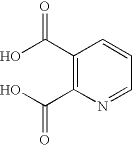
with a base to provide a compound of the Formula

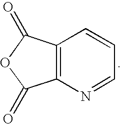

In one specific embodiment the invention provides a method of preparing a compound of Formula (II) 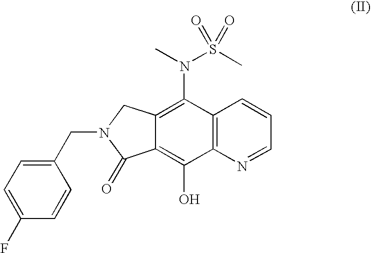
comprising contacting the compound of Formula 8A 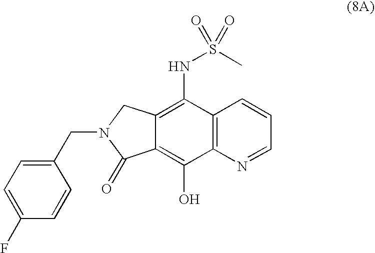
with a methylation reagent.

In one specific embodiment of the invention the methylation reagent is a methyl halide.

In one specific embodiment of the invention the methyl halide is methyl iodide.

In one specific embodiment of the invention the methylation reagent is methyl iodide or dimethylsulfate.

In one specific embodiment the invention further comprises contacting a compound of Formula IA 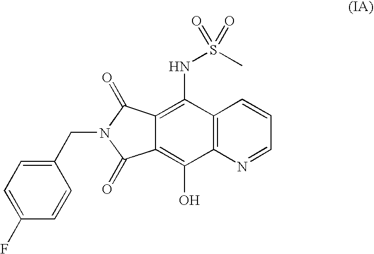
with a reducing agent to provide the a compound of the Formula 8A.

In one specific embodiment of the invention the reducing reagent is a borohydride.

In one specific embodiment of the invention the borohydride is LiBH₄.

In one specific embodiment the invention provides a method of preparing a compound of Formula (II) 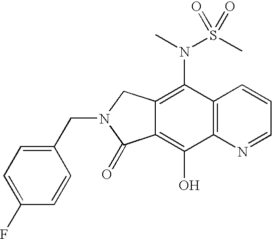
comprising,

a) contacting a compound of Formula IA 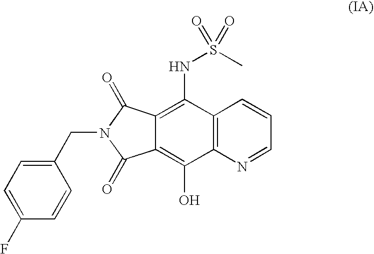
with a reducing agent; and

b) contacting the resultant compound of step a) with a methylation reagent to provide the desired compound.

In one specific embodiment the invention provides a method of preparing a compound of Formula (I) 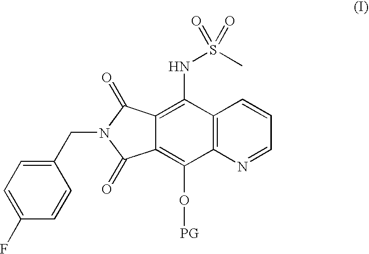
comprising,

a) contacting a compound of the Formula 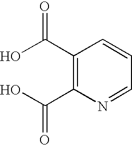
with acetic anhydride to provide a compound of the Formula

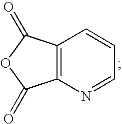

b) contacting the resultant compound of step a) with isopropanol to provide a compound of the Formula

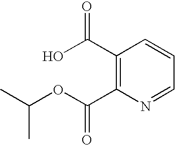

c) contacting the resultant compound of step b) with ammonia and a methylsulfonation reagent to provide a compound of the Formula

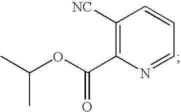

d) contacting the resultant compound of step c) with 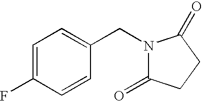
to provide a compound of the Formula

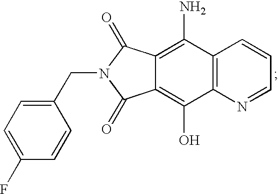

e) contacting the resultant compound of step d) with a protecting agent to provide a compound of the Formula

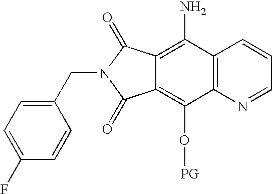

wherein PG is a protecting group;

f) contacting the resultant compound of step e) with a base and methylsulfonation reagent to provide a compound of the Formula

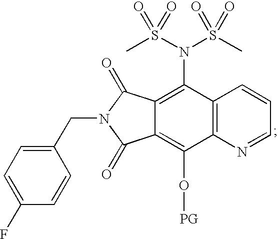

g) contacting the resultant compound of step f) with a base to provide a compound of Formula (I)

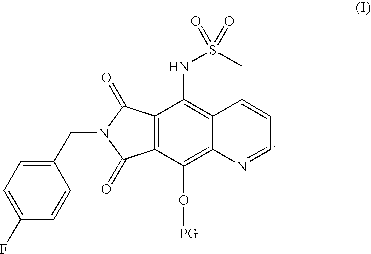

In one specific embodiment the invention further comprises,

h) contacting the resultant compound of step g) with a methylation reagent to provide a compound of the Formula,

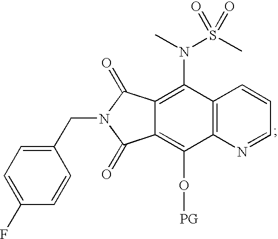

i) contacting the resultant compound of step h) with a first reducing agent to provide a compound of the Formula,

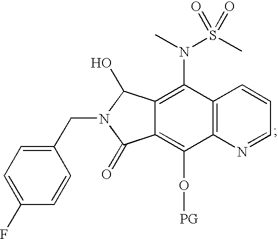

j) contacting the resultant compound of step i) with a second reducing acid and a strong acid to provide the desired compound.

In one specific embodiment the invention further comprises,

h) contacting the resultant compound of step g) with a reducing agent to provide a compound of the Formula,

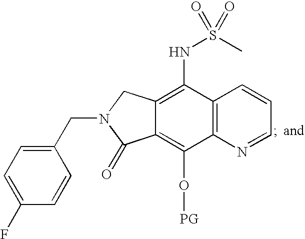

i) contacting the resultant compound of step h) with a methylation agent to provide a compound of Formula (II).

In one specific embodiment the invention further comprises preparing a pharmaceutically acceptable phenolic salt of the Formula 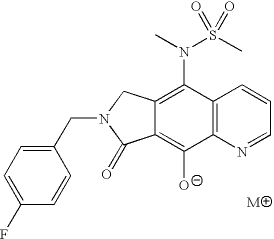
by contacting the compound of Formula (II) 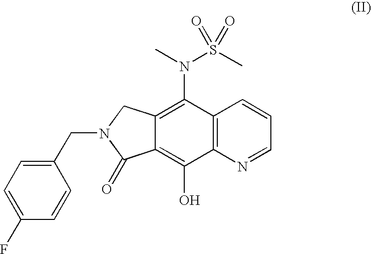
with a base and a solvent or combination of solvents.

In one specific embodiment of the invention the solvent is selected from the group consisting of dimethylformamide, N-methylpyrrolidinone, ethanol, methanol, isopropanol, dimethylacetamide, N-ethylpyrrolidinone, acetone, and methyl tert-butyl ether or combinations thereof.

In one specific embodiment of the invention the combination of solvents is selected from the group consisting of dimethylformamide, N-methylpyrrolidinone, ethanol, methanol, isopropanol, dimethylacetamide, N-ethylpyrrolidinone, acetone, methyl tert-butyl ether.

In one specific embodiment of the invention the base is selected from the group consisting of potassium hydroxide, sodium hydroxide, ethanolamine, ammonium, diethylamine, tromethamine, benzathne, L-lysine, ethylene diamine, deanol, piperazine, 3-(1H-imidazol-1-yl)-1-propanamine, 1,3-diamino-2-propanol, 2-(benzylamino)ethanol, 4-[2-2(4-morpholinyl)ethyl]morphine, dioctylamine, trans 1,4-diaminocyclo-hexane, and 1,2-dimethylaminoethane.

In one specific embodiment of the invention the base is potassium hydroxide.

In one specific embodiment of the invention the solvent is ethanol.

In one specific embodiment the invention further comprises preparing a pharmaceutically acceptable pyridyl salt of the Formula: 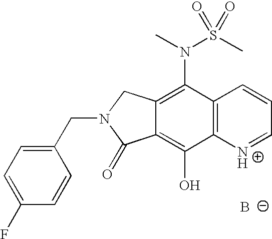
by contacting a compound of Formula (II) 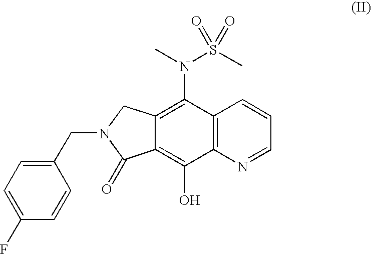
with an acid and a solvent.

In one specific embodiment of the invention the solvent is selected from the group consisting of N-methylpyrrolidinone and ethanol, or combinations thereof.

In one specific embodiment of the invention the combination of solvents are N-methylpyrrolidinone and ethanol.

In one specific embodiment of the invention the acid is selected from the group consisting of hydrochloric acid, methanesulfonic acid, sulfuric acid naphthylenelsulfonic acid, or combinations thereof.

In one specific embodiment the invention provides a compound of the following Formula: 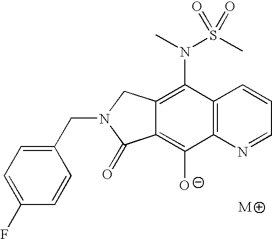
wherein M is sodium or a cation derived from ethanolamine, ammonium, diethylamine, tromethamine, benzathne, L-lysine, ethylene diamine, deanol, piperazine, 3-(1H-imidazol-1-yl)-1-propanamine, 1,3-diamino-2-propanol, 2-(benzylamino)ethanol, 4-[2-2(4-morpholinyl)ethyl]morphine, dioctylamine, trans 1,4-diaminocyclo-hexane, or 1,2-dimethylaminoethane.

In one specific embodiment the invention provides a compound of the following Formula: 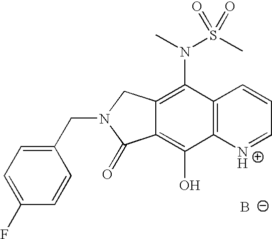
wherein B is chloride, methylsulfonate anion, sulfate, hydrogen sulfate, or naphthylsulfonate anion (i.e. a counterion derived from hydrochloric acid, methanesulfonic acid, sulfuric acid, or naphthylenelsulfonic acid).

In one specific embodiment the invention provides a compound of Formula (III), 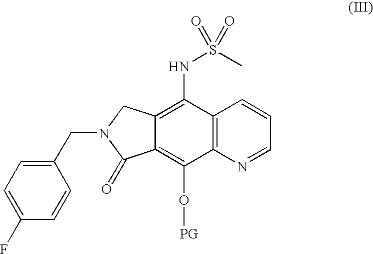
wherein PG is a protecting group (e.g. a silyl protecting group such as tri-isopropylsilyl); or a salt thereof.
Preparation of Compounds of Formula (I) and (II)

The compounds of Formulae I and II can be prepared according to the following non-limiting Reaction Schemes A, B, C and D.

Reaction Scheme A illustrates a preparation of a compound of Formula (I).

Reaction Scheme B illustrates a preparation of a compound of Formula (II) from the compound of Formula (I). In this scheme, the carbonyl at the 6-position of the compound of Formula (I) is reduced to a methylene in one reaction, followed by a combined methylation deprotection reaction to provide the compound of Formula (II).

Reaction-Scheme C illustrates another preparation of the compound of Formula (II) from a compound of Formula (I). In this scheme, the amino functionality at the 5-position of a compound of Formula (I) is methylated followed by a two-step reduction of the carbonyl at the 6-position to provide the compound of Formula (II).

Reaction Scheme D illustrates another preparation of the compound of Formula (II) using the compound of Formula IA, which is obtained from Reaction Scheme A.

Materials prepared according to the reaction schemes may be isolated after each step or taken directly to the next reaction step. There may be advantages in isolating such materials, such as for shipping or storage, or alternatively they may be efficiencies gained by taking the desired material of a given step directly to the step, such as being able to conduct multiple steps in the same reaction vessel without the need for isolation and purification steps.

Starting Materials

Reagents and solvents used in the present invention are available from commercial, such as, Aldrich Chemical Company, 1001 West Saint Paul Avenue, Milwaukee, Wis. 53233 USA.

Reaction Scheme A

Preparation of Compound 2

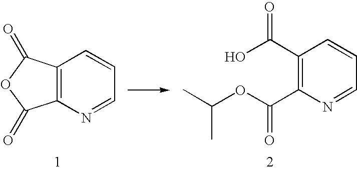

2-(isopropoxycarbonyl)nicotinic acid 2 is prepared by contacting furo[3,4-b]pyridine-5,7-dione 1 with isopropanol and refluxing for about 12 to 24 hours, (e.g. about 18 hours) following conditions disclosed in Dunn, A. D.; Mills, M. J.; Henry, W. Org. Prep. Proced. Int. 1982, 14, 396-399. The starting material, furo[3,4-b]pyridine-5,7-dione 1 also known as 2,3-pyridinedicarboxylic anhydride, is commercially available from Aldrich Chemical Company, Inc. 1001 West Saint Paul Avenue, Milwaukee, Wis. 53233. The desired compound 2 was isolated using chromatographic procedures known to those of ordinary skill in the art.
Preparation of Compound 3

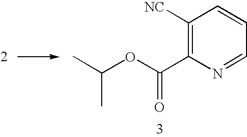

2-(isopropoxycarbonyl)nicotinic acid 2 was treated with a reactive sulfonate ester reagent, such as, an alkylsulfonyl chloride (e.g. methanesulfonyl chloride) to obtained isopropyl 3-cyanopicolinate 3 following disclosures from Dunn, A. D.; Mills, M. J.; Henry, W. Org. Prep. Proced. Int. 1982, 14, 396-399.
Preparation of Compound 4

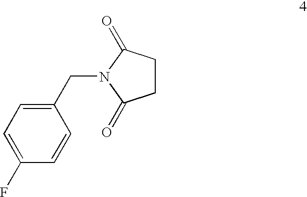

1-(4-Fluorobenzyl)pyrrolidine-2,5-dione 4 is prepared from succinimide and 4-fluorobenzyl bromide following procedures similar to those described in International Patent Application Publication Number WO 2004/035576.
Preparation of Compound 5

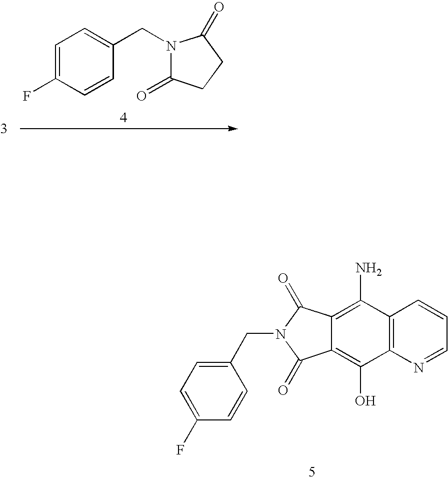

7-(4-Fluorobenzyl)-5-amino-9-hydroxy-7H-pyrrolo[3,4-g]quinoline-6,8-dione hydrochloride 5 is prepared by contacting about 1 equivalent of compound 3 with about 1 equivalent of compound 4 in an organic solvent (e.g. a moderately polar solvent, such as THF) in a flask at a temperature of about 0° C. To this mixture is added and about 2.5 equivalent of a base (e.g. a strong base, such as NaHMDS (sodium hexamethyldisilazane)) in an organic solvent (e.g. a moderately polar solvent, such as THF) in a gradual (e.g. dropwise) manner over a period of about 5 to 15 minutes (e.g. about 10 minutes). Following the addition, the reaction solution stirred for a period of about 30 minutes to 90 minutes (e.g. about 60 minutes/1 hour), and is allowed to warm to ambient temperature. The reaction solution is then cooled to about 0° C. and quenched with an acid (e.g. a strong acid, such as 6N HCl). The desired product, compound 5 is obtained as a salt, using crystallization procedures known to those of ordinary skill in the art.
Preparation of Compound 6

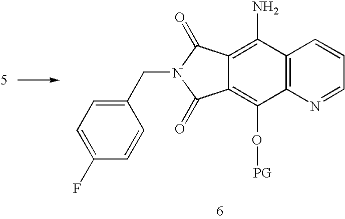

About 1 equivalent of compound 5 is combined in a flask with an organic solvent (e.g. an aprotic polar solvent, such as DMF) followed by about 2 to 4 equivalent of a base (e.g. an amine base, such as triethylamine), (e.g. about 3 equivalents of triethylamine, and about 0.1 equivalents of DMAP (4-(dimethylamino)pyrdine)). To this solution was added about 1.0 to 2.0 equivalents of a trialkylsilyl halide, such as trimethylsilyl halide, hexamethyldisilazane, or t-butyldimethylsilyl halide (e.g. about 1.2 equivalents of triisopropylsilyl chloride) dissolved in DMF. Following the completion of addition, the reaction mixture was stirred at about room temperature for about 30 to 90 minutes (e.g. about 1 hour). The reaction mixture was subsequently diluted with an organic solvent, such as EtOAc and quenched with water. Compound 6 is obtained following standard extraction processing using chromatographic procedures known to those of ordinary skill in the art.
Preparation of Compound 7

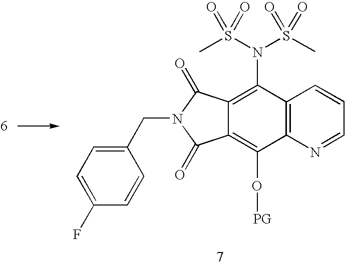

Compound 6 is combined with an organic solvent (e.g. an apolar solvent, such as dichloromethane), and about 4 to 12 equivalents (e.g. about 8 equivalents) of a base (e.g. an amine base such as triethylamine). The resulting solution is cooled to about 0 to −10° C. (e.g. about −2 to −5° C.) under an inert atmosphere. To this solution is added about 2 to 6 equivalents of a sulfonylating agent such as alkylsulfonyl halide, alkylsulfonic anhydride (e.g. methanesulfonic anhydride), or alkylsulfonic acid/thionyl chloride (e.g. MsCl) in a dropwise manner. Following addition, the reaction mixture is agitated for a period of about 30 to 120 minutes (e.g. about 1 hour) at a temperature in the range of about 5 to −5° C. (e.g. around 0° C.). An aqueous salt solution is prepared (e.g. NH₄Cl,) and kept at a temperature below 5° C. The progress of the reaction is monitored using suitable assaying methods (e.g. by HPLC. Following completion of the reaction, the aqueous salt solution is combined with the reaction mixture and rinsed with a suitable solvent and water. The combined mixture is agitated and allowed to settle to separate into aqueous and organic layers. The desired compound 7, wherein PG is a protecting group, is obtained from organic layer using chromatographic procedures known to those of ordinary skill in the art.
Preparation of a Compound of Formula (I)

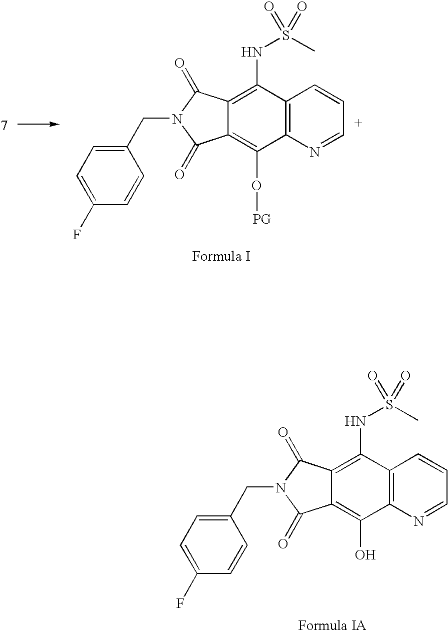

A solution of compound 7 in an organic solvent (e.g. a aprotic solvent, such as THF is cooled to a temperature in the range of about 5 to −5° C. (e.g. in the range of about 0 to −5° C.). To this solution is added about 1 to 2 equivalents of a base such as NaOH, NaH, potassium carbonate, sodium ethoxide/NaOH, ammonia, or tetrabutylammonium fluoride (e.g. about 1.8 equivalents of potassium t-butoxide), at a rate so as to maintain a solution temperature of below about 10° C. A separate aqueous salt solution is prepared (e.g. NH₄Cl) and cooled to a temperature below 5° C. The progress of the reaction is monitored using suitable assaying methods (e.g. by HPLC). When the reaction is judged complete, the aqueous solution is combined with the reaction mixture. The combined solution is then rinsed with an organic solvent and water. The desired compound of Formula (I) is obtained from the organic layer using chromatographic procedures known to those of ordinary skill in the art. Also obtained is the compound of Formula IA, which is the compound of Formula (I) where the protecting group has been removed.

Reaction Scheme B

Preparation of Compound 8

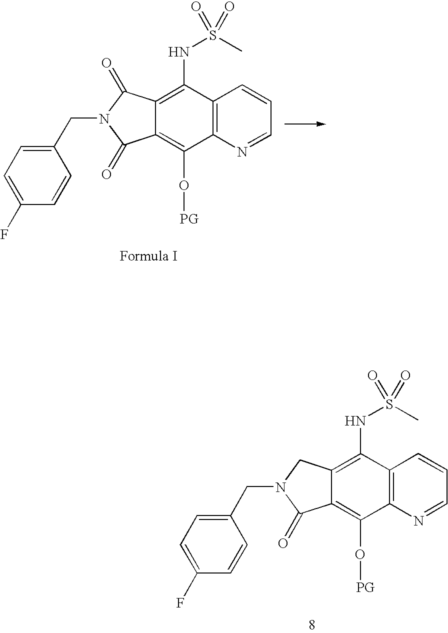

A slurry is formed by combining the compound of Formula (I) with an organic solvent (e.g. a polar aprotic solvent, such as THF). The slurry is cooled to a temperature in the range of about 5 to −15° C. under an inert atmosphere. To the reaction mixture is added about 1 or more equivalents of a reducing agent, such as, aluminum based reagents (LiAlH[OC(CH₃)₃]₃, Red-Al®, [(CH₃)₂CHCH₂]₂AlH, NaAlH₄, LiAlH₄), boron based reagents (LiBHEt₃, Lithium 9-BBN hydride, L-selectride, K-selectride, KS-selectride, LS-selectride, N-selectride, NaBH₄, catecholborane, borane and complexes thereof such as dimethylsulfide, ammonia, dimethylamine, diphenylphosphine, isoamylsulfide, morpholine (polymerbound or other), N,N-diethylaniline, N,N-diisopropylethylamine, pyridine, tert-butylamine, tetrahydrofuran, tributylphosphine, triethylamine, trimethylamine, and triphenylphosphine and reductants made from these borane complexes such as lithium aminoborohydrides) silane based reagents in conjunction with a strong acid (trialkylsilanes, triarylsilanes, and alkylarylsilanes), (e.g. about 1.1 equivalents of LiBH₄) over a period of about 1 to 3 hours (e.g. about 1.5 hours). The reaction mixture is heated to reflux and maintained for a period of about 30 minutes to 2 hours (e.g. about 1 hour). After refluxing, about 1.1 equivalent of an alcohol (e.g. methanol) is added to the reaction mixture over a period of about 30 minutes to 2 hours (e.g. about 1.5 hours). The reaction mixture is maintained at reflux for a period of about 30 minutes to 2 hours (e.g. 1 hour). After refluxing, about 1.1 equivalents of methanol is added to the reaction mixture over a period of about 30 minutes to 2 hours (e.g. about 1.5 hours). The progress of the reaction is monitored using any suitable assaying technique (e.g. ¹⁹F NMR). Upon completion of the reaction, about 2 to 4 equivalents (e.g. about 2.9 equivalents) of methanol is added to the reaction mixture. The reaction mixture is cooled to a temperature in the range of about 19 to 25° C. To the reaction mixture an organic solvent, (e.g. isopropyl acetate), and an aqueous solution (e.g. NH₄Cl) is combined with the reaction mixture. The combined solution is agitated and the aqueous and organic layers are allowed to form. The desired compound 8 is obtained from the organic layer using crystallization procedures known to those of ordinary skill in the art, and using suitable assaying methods to monitor the purity of the product.
Preparation of Formula (II)

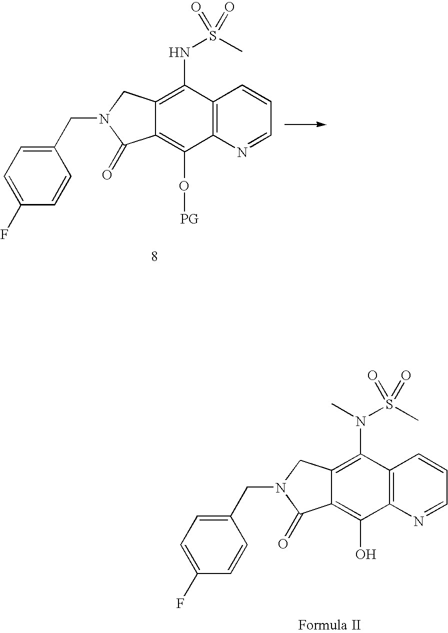

A slurry is formed by combining compound 8 with an organic solvent at a ratio of about 1.8 mL of solvent to grams of compound 8 (e.g. acetone). The slurry is cooled to a temperature in the range of 15 to 20° C. and kept under an inert atmosphere. To this slurry is added about 1 to 2 equivalents of a base (e.g. a strong base, for example, about 1.1 equivalents of potassium hydroxide), at a rate to maintain the temperature below 25° C. After completion of the addition, the mixture is cooled to a temperature in the range of about 15 to 20° C. About 1 to 2 equivalents (e.g. about 1.1 equivalents) of a methylation reagent (e.g. dimethylsulfate) is added at a rate to maintain the temperature at about below 25° C. Upon completion of the addition, the mixture is agitated for about 30 minutes to 2 hours (e.g. about 40 minutes) without external cooling. The progress of the reaction is monitored by any assaying technique that is suitable (e.g. by HPLC). Upon completion of the reaction, MeOH is added to the mixture at a ratio of about 2.6 mL per gram of starting material (compound 8), followed by about 0.25 to 1 equivalent of TFA (e.g. about 0.50 equivalent). The resultant slurry is heated (e.g. to about 30° C. or to about 50° C.). The progress of the reaction is monitored. Upon completion of the reaction, the mixture is diluted by the addition of water at a temperature in the range of about 35 to 50° C. (e.g. about 42° C.). The mixture is allowed to cool with agitation over time. After about 10 to 20 hours (e.g. about 15 hours), the slurry is filtered and washed at least once with an alcohol (e.g. MeOH). The resultant cake is dried in an oven at about 50° C. under vacuum (approximately 30 inches Hg) for a period of about 2 to 5 hours (e.g. about 3 hours). Following the drying, the cake was combined with an alcohol (e.g. MeOH) at a ratio of about 11.7 mL to grams of material; and a second organic solvent (e.g. an aprotic polar solvent such as acetonitrile or acetone) at a ratio of about 3.3 mL to grams of material. The combined mixture is then refluxed with agitation for about 2 hours. The slurry is allowed to cool to room temperature and agitated for about 12 to 20 hours (e.g. about 16 hours). The slurry is again washed with an alcohol and dried under vacuum, as was previously described. After about 24 hours, the desired product is obtained.

Reaction Scheme C

Preparation of Compound 9

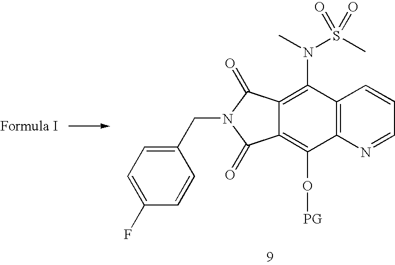

A solution of the compound of Formula (I) is dissolved in an organic solvent (e.g. an aprotic polar solvent, such as DMF) and cooled to a temperature in the range of about −20 and 15° C. (e.g. in the range of about −10 and 5° C.). To this solution is added about 1 to 2 equivalents of a base (e.g. about 1.5 equivalents of potassium carbonate). A separate solution of about 1 to 2 equivalents of an alkylating agent such as alkyl halide, dialkylsulfate (for example, dimethylsulfate), dimethylcarbonate, phenyltrimethylammonium halide, methylmethanesulfonate, trimethyloxonium tetrafluoroborate, trimethylsulfonium halide or utilizing a phase transfer system (e.g. a methyl-halide, such as 1.5 equivalents of methyl iodide) in an organic solvent (e.g. an aprotic polar solvent, such as DMF) is prepared and cooled to a temperature in the range of about −10 to 10° C. (e.g. about 0° C.). The methyl halide solution is added to the reaction mixture while maintaining a temperature below about 5° C. The progress of the reaction is monitored, and when it is found to be complete, the reaction mixture is allowed to warm to a temperature of about 20° C. The reaction mixture is worked up by addition of a suitable organic solvent, such as isopropyl acetate, and aqueous solution, such as NH₄Cl, and water. The combined solution is agitated. The aqueous and organic layers are then allowed to form. The organic layer is isolated and is washed with an aqueous solution, such as water. The washing process is repeated with brine. A compound of formula 9 is crystallized from the organic layer using crystallization techniques known to those of ordinary skill in the art.
Preparation of the Compound 10

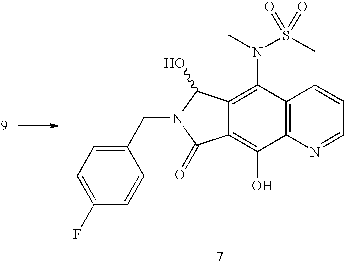

A solution is formed by combining compound 9 with an organic solvent (e.g. an aprotic solvent such as THF) at a ratio of about 3.9 mL per gram of starting material. The resulting solution is cooled to a temperature of about 5° C. under an inert atmosphere. About 1 to 3 equivalents of a reducing agent, such as, aluminum based reagents (LiAlH[OC(CH₃)₃]₃, Red-Al®, [(CH₃)₂CHCH₂]₂AlH, NaAlH₄, LiAH₄), boron based reagents (LiBHEt₃, Lithium 9-BBN hydride, L-selectride, K-selectride, KS-selectride, LS-selectride, N-selectride, NaBH₄, catecholborane, borane and complexes thereof such as dimethylsulfide, ammonia, dimethylamine, diphenylphosphine, isoamylsulfide, morpholine (polymerbound or other), N,N-diethylaniline, N,N-diisopropylethylamine, pyridine, tert-butylamine, tetrahydrofuran, tributylphosphine, triethylamine, trimethylamine, and triphenylphosphine and reductants made from these borane complexes such as lithium aminoborohydrides) silane based reagents in conjunction with a strong acid (trialkylsilanes, triarylsilanes, and alkylarylsilanes), (e.g. about 2 equivalents of LiBH₄) in an organic solvent (e.g. an aprotic solvent such as THF) is added to the reaction mixture at a rate such that the temperature of the mixture is kept at about below 15° C. The mixture is cooled to about −10° C. and a solution of MeOH (about 4 to 10 equivalents (e.g. about 7 equivalents) in an organic solvent (e.g. an aprotic solvent such as THF) is slowly added to the reaction mixture to activate the reducing agent. The rate of addition is adjusted to maintain the temperature of the solution about below 5° C. The reaction progress is monitored by the formation of the diastereomers of compound 10 using any suitable assaying technique (e.g. HPLC). After completion of the reaction, the remaining reducing agent is quenched by the addition of an aqueous salt solution (e.g. NH₄Cl). The mixture is worked up using a succession of water rinses followed by a brine rinse. The organic layer is isolated from the mixture and concentrated.

Preparation of Compound of Formula (II)

A solution is formed by combining compound 10 with about 5 to 15 equivalents (e.g. about 10 equivalents) of triethylsilane in a nonpolar organic solvent (e.g. dichloromethane). An acid (e.g. a strong acid, such as TFA) is added to the reaction mixture. The progress of the reaction is monitored using any suitable assaying technique (e.g. HPLC). The progress of the reaction is determined by the disappearance of the diastereomers of 10. Upon completion of the reaction, MeOH at a ratio of about 4.5 mL per gram of starting material, compound 10, is added to the solution. The progress of this reaction is again monitored as before. Upon completion of the reaction, the organic phase was concentrated and separated from the aqueous phase. The desired product, the compound of Formula (II) was isolated from the organic phase using chromatographic techniques known to those of ordinary skill in the art.

Reactions Scheme D

Preparation of Compound 8A

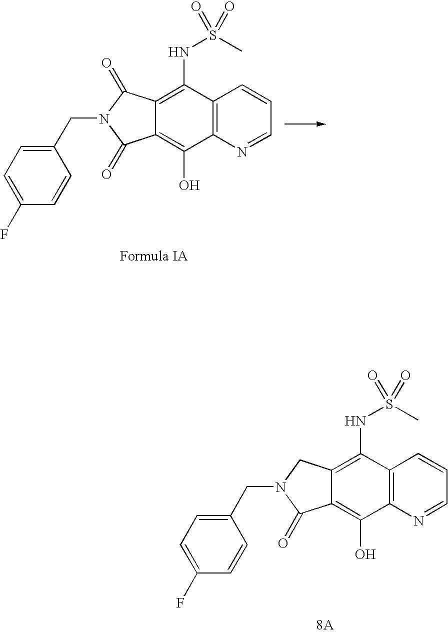

A slurry is formed by combining the compound of Formula (I) with an organic solvent (e.g. an aprotic solvent, such as THF). The slurry is cooled to a temperature in the range of about 5 to −15° C. under an inert atmosphere. To the reaction mixture is added about 1 or more equivalents of a reducing agent, such as, aluminum based reagents (LiAlH[OC(CH₃)₃]₃, Red-Al®, [(CH₃)₂CHCH₂]₂AlH, NaAlH₄, LiAlH₄), boron based reagents (LiBHEt₃, Lithium 9-BBN hydride, L-selectride, K-selectride, KS-selectride, LS-selectride, N-selectride, NaBH₄, catecholborane, borane and complexes thereof such as dimethylsulfide, ammonia, dimethylamine, diphenylphosphine, isoamylsulfide, morpholine (polymerbound or other), N,N-diethylaniline, N,N-diisopropylethylamine, pyridine, tert-butylamine, tetrahydrofuran, tributylphosphine, triethylamine, trimethylamine, and triphenylphosphine and reductants made from these borane complexes such as lithium aminoborohydrides) silane based reagents in conjunction with a strong acid (trialkylsilanes, triarylsilanes, and alkylarylsilanes), (e.g. about 4.0 equivalents of LiBH₄) in a dropwise fashion. The reaction mixture is warmed to a temperature in the range of about 30° C. (e.g. about 60° C.) and stirred for a period of about 8 to 24 hours (e.g. about 16 hours). MeOH is added to the reaction mixture followed by about 0.5 to 2 equivalents of an acid (e.g. a strong acid, such as about 0.67 equivalents of TFA). The desired compound 8A is obtained from the reaction mixture by crystallization using suitable assaying methods to monitor the purity of the product.
Preparation of a compound of Formula (II)

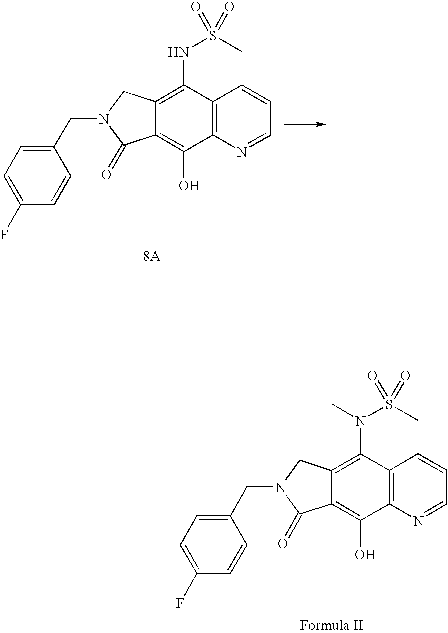

A slurry is formed by combining compound 8A with an organic solvent at a ratio of about 8 to 16 mL of solvent to gram of starting material (e.g. about 12 mL of acetone per gram of starting material). The resultant mixture is kept under an inert atmosphere. To this mixture is added about 1 to 5 equivalents of a base (e.g. a strong base, such as about 3 equivalents of potassium hydroxide), in a dropwise manner. After completion of the addition, about 1 to 5 equivalents (e.g. about 3 equivalents of a methylation reagent, such as dimethylsulfate) is added to the mixture. The progress of the reaction is monitored by any assaying technique that is suitable (e.g. HPLC). Upon completion of the reaction, the desired compound of Formula (II) is obtained using chromatographic and/or crystallization techniques known to those of ordinary skill in the art.
Preparation of Phenolic Salts of Formula (II)

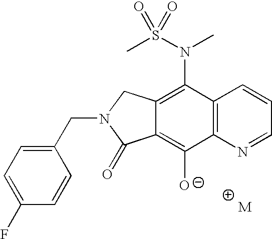

The phenolic salt of a compound of Formula (II) can be prepared as described below. For example, a solution is formed by combining a compound of Formula (II) with an appropriate solvent or solvent system including but not limited to alcohols, such as MeOH, EtOH, iPrOH, t-BuOH; ethers, such as THF, 2-methyl THF, tert-butyl ether (MTBE); ketones, such as methylisobutyl ketone (MIBK), methyl, acetone); amides, such as NMP, NEP, DMF, dimethylacetamide; or toluene (e.g. dimethylformamide, N-methylpyrrolidinone, ethanol, methanol, isopropanol, dimethylacetamide, N-ethylpyrrolidinone, acetone, methyl tert-butyl ether are used). The resulting solution or mixture was agitated at a temperature in the range of about between 20 to 80° C. An appropriate base was added and the resulting mixture was agitated at a temperature range of about between 20 to 80° C. for a period of time. The resulting salt was isolated and rinsed with an appropriate solvent or solvent system. The solid was dried in vacuo at a temperature range of about between 20 to 80° C. for a period of time, yielding the desired salt.
Preparation of Pyridyl Salts of Formula (II)

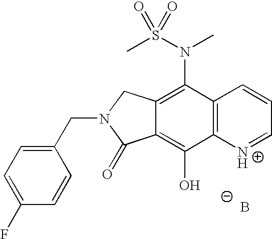

The pyridyl salt of the compound of Formula (II) is readily obtained as follows. For example, a solution is formed by combining a compound of Formula (II) with an appropriate solvent or solvent system including but not limited to alcohols, such as EtOH; and amides, such as NMP (N-methyl-2-pyrrolidinone). The resulting solution or mixture is agitated at a temperature in the range of about between 20 to 80° C. An appropriate acid is added to the solution and the resulting mixture is further agitated at a temperature in range of about between 20 to 80° C. for a period of time. The resulting salt is isolated and rinsed with an appropriate solvent or solvent system. The solid is dried in vacuo at a temperature in the range of about between 20 to 80° C. for a period of time, yielding the desired salt.

### Specific Compounds

A Compound of Formula (I), 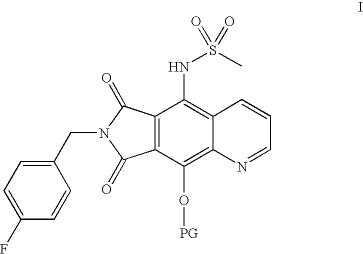
wherein, PG is a protecting group; and a compound of Formula (II), N-(7-(4-fluorobenzyl)-9-hydroxy-8-oxo-7,8-dihydro-6H-pyrrolo[3,4-g]quinolin-5-yl)-N-methylmethanesulfonamide, 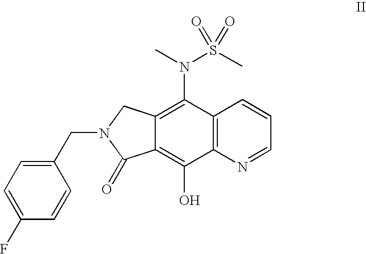
can be prepared using the synthetic processes of the invention. The compound of Formula (I) is a synthetic intermediate that can be used to prepare an integrase inhibitor of Formula (II).

### EXAMPLES

The following preparations and examples are given to enable those skilled in the art to understand and practice the present invention. The examples should not be construed as limiting the scope of the invention, but as illustrative and representative thereof.

### Example 1

### Preparation of 2-(isopropoxycarbonyl)nicotinic acid

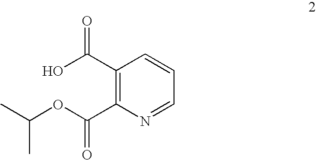

Furo[3,4-b]pyridine-5,7-dione (39.1 g, 322.5 mmol) was suspended in isopropanol (250 mL, 1.3 M) and refluxed for 18 h. The reaction mixture was filtered and concentrated to afford 2-(isopropoxycarbonyl)-nicotinic acid 2 (54.21 g, 99%) as a tan solid: ¹H NMR (DMSO-d6, 300 MHz) 8.69 (d, 1H), 8.21 (d, 1H), 7.58 (m, 1H), 5.10 (m, 1H), 1.23 (d, 6H). (Taken from: Dunn, A. D.; Mills, M. J.; Henry, W. Org. Prep. Proced. Int. 1982, 14, 396-399.)

### Example 2

### Preparation of Isopropyl 3-cyanopicolinate

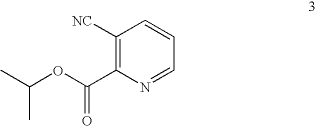

2-(isopropoxycarbonyl)nicotinic acid (36.7 g, 175 mmol) in pyridine (575 mL, 0.3 M) was cooled to 0° C. and treated with MsCl (13.5 mL, 175 mmol) under Ar. After 1 hour, NH₃ was bubbled through the reaction mixture for 5 min and the solution was warmed to room temperature. Residual NH₃ was removed in vacuo and the reaction mixture was cooled to 0° C. and treated with MsCl (115 mL, 1490 mmol). After 12 h, solvent was removed and the mixture was dissolved in saturated NaHCO₃ (500 mL) and stirred for 1 h. The solution was filtered and extracted with Et₂O (2×500 mL). The organic layers were combined, washed with saturated NaHCO₃ (3×150 mL) and brine (150 mL), dried (Na₂SO₄), and concentrated. Flash chromatography (SiO₂, 10×33 cm, 0-100% EtOAc-hexanes gradient) afforded the desired isopropyl 3-cyanopicolinate 3 (21.7 g, 65%) as an orange solid: R_{f}=0.74 (20% MeOH-CH₂Cl₂); ¹H NMR (CDCl₃, 300 MHz) 8.89 (d, 1H), 8.10 (d, 1H); 7.56 (m, 1H), 5.37 (m, 1H), 1.43 (d, 6H); ¹³C NMR (CDCl₃, 75 MHz) 162.4, 152.1, 149.8, 142.9, 126.4, 115.3, 110.1, 71.1, 20.6; MS (ESI) m/z 191 [M+H]⁺. (Taken from: Dunn, A. D.; Mills, M. J.; Henry, W. Org. Prep. Proced. Int. 1982, 14, 396-399.)

### Example 3

### Preparation of 1-(4-fluorobenzyl)pyrrolidine-2,5-dione

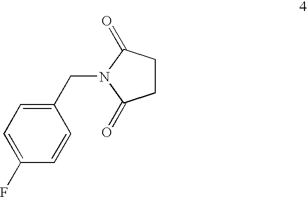

Succinimide (20 g, 202 mmol) in acetone (500 mL, 0.375 M) was treated with K₂CO₃ (84 g, 605 mmol) and stirred for 5 minutes. 4-Fluorobenzyl bromide (28 mL, 222 mmol) was added and the reaction was mechanically stirred at reflux overnight. The mixture was filtered and the solvent was removed. The remaining solid was filtered and washed with hexanes (5×100 mL) to afford 1-(4-fluorobenzyl)-pyrrolidine-2,5-dione 4 (37.8 g, 91%) as a white solid: R_{f}=0.33 (50% EtOAc-hexanes); ¹H NMR (CDCl₃, 300 MHz) 7.33 (m, 2H), 6.91 (m, 2H), 4.56 (s, 2H), 2.65 (s, 4H).

### Example 4

### Preparation of 7-(4-fluorobenzyl)-5-amino-9-hydroxy-7H-pyrrolo[3,4-g]quinoline-6,8-dione hydrochloride

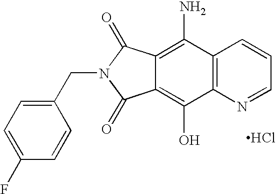

Into a flask containing isopropyl 3-cyanopicolinate (10 g, 52.6 mmol, 1 equiv), as prepared in Example 2 and 1-(4-fluorobenzyl)-pyrrolidine-2,5-dione (11.98 g, 57.88 mmol, 1.1 equiv) was added THF (170 mL, 0.3 M). The flask was cooled to 0° C. and NaHMDS (131 mL, 131 mmol, 2.5 equiv, 1 M THF) diluted in THF (90 mL) was added dropwise via an addition funnel over 10 min. The ice-bath was removed and the reaction allowed to stir for an hour. The flask was cooled to 0° C. and slowly quenched with HCl (6 N, 55 mL) before being concentrated in vacuo to a red paste. Ethyl ether (400 mL) was added to the flask along with water (50 mL). It was allowed to stir vigorously for 15 min before being filtered over a sintered funnel. The red residue was washed with water (2×15 mL) and ether (3×50 mL) and allowed to air dry in a vacuum oven at 65° C. for several hours to afford 7-(4-fluorobenzyl)-5-amino-9-hydroxy-7H-pyrrolo[3,4-g]quinoline-6,8-dione hydrochloride, the hydrochloride salt of compound 5 (16.7 g, 95%) of a red powder was obtained. ¹H NMR (DMSO-d₆, 300 MHz) 8.96 (d, 2H), 7.77 (m, 1H), 7.29 (m, 2H), 7.12 (m, 2H), 6.63 (br s, 2H), 4.65 (s, 2H); MS (ESI) m/z 338 [M+H]⁺.

### Example 5

### Preparation of 7-(4-fluorobenzyl)-5-amino-9-(triisopropylsilyloxy)-7H-pyrrolo[3,4-g]quinoline-6,8-dione

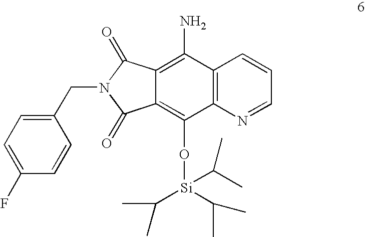 [0143] wherein PG is triisopropylsilyl

Into a flask containing 7-(4-fluorobenzyl)-5-amino-9-hydroxy-7H-pyrrolo[3,4-g]quinoline-6,8-dione (40 g, 119 mmol, 1 equiv) was added DMF (520 mL, 0.24 M) followed by TEA (49.6 mL, 142 mmol, 3 equiv) and DMAP (1.45 g, 11.86 mmol, 0.1 equiv). TIPSCl (30.2, 142 mmol, 1.2 equiv) was dissolved in DMF (70 mL) and added dropwise to the reaction flask via an addition funnel over 20 min before being allowed to stir at room temperature for 1 h. The reaction mixture was diluted with EtOAc (1.2 L) and quenched with water (600 mL) before separating the layers. The aqueous layer was extracted with EtOAc (2×500 mL). The organic layers were combined and washed with water (3×600 mL), citric acid (10%, 600 mL), brine (600 mL) before being dried over Na₂SO₄, filtered and concentrated in vacuo to yield a crude solid (lime green, 81.1 g). This solid was washed and filtered on sintered funnel with hexanes (5×300 mL) to afford 7-(4-fluorobenzyl)-5-amino-9-(triisopropylsilyloxy)-7H-pyrrolo[3,4-g]quinoline-6,8-dione, compound 6 wherein PG is triisopropylsilyl, (48.3 g, 82%) as a light yellow solid. R_{f}=0.63 (50% EtOAc-hexanes): ¹H NMR (CDCl₃, 300 MHz) 8.87 (d, 1H), 8.19 (d, 1H), 7.47 (m, 1H), 7.40 (m, 2H), 6.95 (t, 2H), 5.70 (br s, 2H), 4.77 (s, 2H), 1.45 (m, 3H), 1.07 (d, 18H); MS (ESI) m/z 494 [M+H]⁺.

### Example 6

### Preparation of N-(7-(4-fluorobenzyl)-6,8-dioxo-9-(triisopropylsilyloxy)-7,8-dihydro-6H-pyrrolo[3,4-g]quinolin-5-yl)-N-(methylsulfonyl)methanesulfonamide

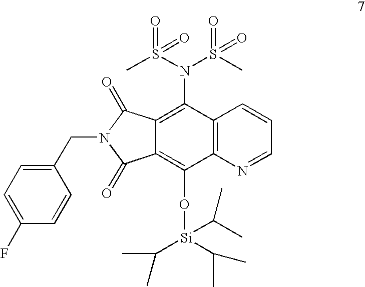 [0146] wherein PG is triisopropylsilyl

7-(4-Fluorobenzyl)-5-amino-9-(triisopropylsilyloxy)-7H-pyrrolo[3,4-g]quinoline-6,8-dione (811.7 g, 1.64 mol) was charged into a 22-L reaction flask. Dichloromethane (8 L) and triethylamine (1.83 L, 13.16 mol, 8 equivalents) were charged and the resulting solution was cooled to an internal temperature of −2 to −5° C. under nitrogen atmosphere. A solution of methanesulfonyl chloride (0.511 L, 6.58 mol, 4 equiv) in dichloromethane (8 L) was prepared and charged into an addition funnel. Methanesulfonyl chloride in dichloromethane was charged slowly into the reaction content at a rate as to maintain the internal temperature <5° C. The addition time for charging half the amount of MsCl in DCM was 45 to 50 minutes. The addition time of the second portion of MsCl in DCM was 30 minutes. Upon completion of addition, the reaction was agitated for 1 hour at <0° C. Reaction progress was monitored by HPLC assay. In a separate reaction vessel was charged saturated aqueous NH₄Cl solution (4 L) and water (4 L). The mixture was agitated and cooled to <5° C. Once the reaction was judged complete by HPLC assay, the reaction content was charged in the diluted aqueous NH₄Cl solution and rinsed forward with DCM (0.5 L) and water (2×1 L). The internal temperature was 0.9° C. and allowed to warm slightly. The jacket temperature was kept at <35° C. This process yielded N-(7-(4-fluorobenzyl)-6,8-dioxo-9-(triisopropylsilyloxy)-7,8-dihydro-6H-pyrrolo[3,4-g]quinolin-5-yl)-N-(methylsulfonyl)methane-sulfonamide, compound 7, wherein PG is triisopropylsilyl, with a theoretical yield of 1.07 Kg (100%).

Analytical data: R_{f}=0.30 (THF/hexanes=1/2); ¹H NMR (CDCl₃, 300 MHz) 8.98 (dd, 1H, J=4.2 and 1.5 Hz), 8.53 (dd, 1H, J=8.4 and 1.5 Hz), 7.74 (dd, 1H, J=8.4 and 4.2 Hz), 7.47 (appt dd, 2H, J=8.7 and 5.4 Hz), 7.02 (appt t, 2H, J=8.7 Hz), 4.86 (s, 2H), 3.56 (s, 6H), 1.55 (m, 1H, J=7.5 Hz) 1.13 (d, 18H, J=7.5 Hz); MS (ESI) m/z 650 [M+H]⁺

### Example 7

### Preparation of N-(7-(4-fluorobenzyl)-6,8-dioxo-9-(triisopropylsilyloxy)-7,8-dihydro-6H-pyrrolo[3,4-g]quinolin-5-yl)methanesulfonamide

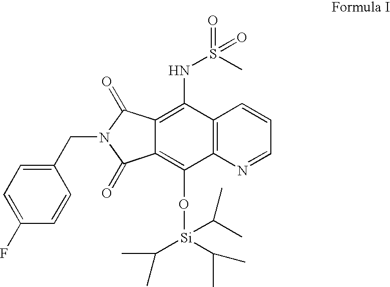 [0150] wherein PG is triisopropylsilyl

A solution of N-(7-(4-fluorobenzyl)-6,8-dioxo-9-(triisopropylsilyloxy)-7,8-dihydro-6H-pyrrolo[3,4-g]quinolin-5-yl)-N-(methylsulfonyl)-methanesulfonamide (˜1.07 kg, 1.64 mol) in 2 L of THF was cooled to an internal temperature of 0° C. to −5° C. The solution was charged 1.0 M solution of potassium t-butoxide in THF (3.02 L, 3.02 mol, 1.84 equiv) to the reaction content at a rate as to maintain content temperature <10° C. The reaction was monitored by HPLC. A separate reaction vessel was charged saturated aqueous NH₄Cl solution (4 L) and water (4 L). The mixture was agitated and cooled to <5° C. Once the reaction was completed by HPLC assay, the reaction content was charged into the diluted aqueous NH₄Cl solution, and rinsed forward with DCM (0.5 L) and water (2×1 L). Subsequently, the mixture was charged a second time with additional DCM (16 L) and mixed well. The mixture was agitated and allowed to settle for at least 5 hours. The organic layer, which contained the product was collected and dried over Na₂SO₄. The slurry was filtered and the solid layer rinsed with DCM (1 L). The organic layer was then concentrated via vacuum distillation to a crude oil. This layer was charged in dichloromethane (2 L) to re-dissolve crude oil. A silica gel pad was prepared by slurrying SiO₂ (6 kg) in a 1:1 solution of ethyl acetate/heptane (16 L) and 0.05% triethylamine and loaded into an appropriate filter. A filter cloth was placed on top of the silica gel layer. The product solution in DCM was charged onto the silica gel pad and eluted with 1:1 solution of ethyl acetate/heptane (30 L) followed 100% ethyl acetate. The solvent was removed by vacuum distillation. Near the end of distillation, precipitation was observed. Residual ethyl acetate was removed by charging additional heptane (1-2 L) for co-evaporation. Heptane (3 L) was charged-into the solution to suspend product solid. The contents were cooled to 20-25° C. The products were isolated by filtration and the resulting solids were rinsed thoroughly with heptane (5×1 L). The solids were dried in a vacuum oven at ambient temperatures.

The two-step process of Example 6 and the present example yielded N-(7-(4-fluorobenzyl)-6,8-dioxo-9-(triisopropylsilyloxy)-7,8-dihydro-6H-pyrrolo[3,4-g]quinolin-5-yl)methanesulfonamide, a compound of Formula (I), wherein PG is triisopropylsilyl. Analytical data: R_{f}=0.40 (THF/hexanes=1/2); ¹H NMR (CDCl₃, 300 MHz) 8.95 (s, 1H), 8.92-8.94 (m, 1H), 7.62-7.68 (m, 2H), 7.46 (appt dd, 2H, J=8.7 and 5.1 Hz), 7.02 (appt t, 2H, J=8.7 Hz), 4.84 (s, 2H), 3.02 (s, 3H), 1.54 (m, 1H, J=7.2 Hz) 1.12 (d, 18H, J=7.2 Hz); MS (ESI) m/z 570 [M−H]⁻

### Example 8

### Preparation of N-(7-(4-fluorobenzyl)-8-oxo-9-(triisopropylsilyloxy)-7,8-dihydro-6H-pyrrolo[3,4-g]quinolin-5-yl)methanesulfonamide

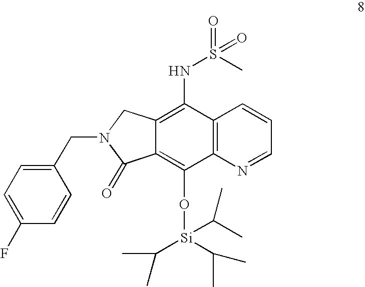 [0154] wherein PG is triisopropylsilyl

Into a 12 L reaction flask was charged N-(7-(4-fluorobenzyl)-6,8-dioxo-9-(triisopropylsilyloxy)-7,8-dihydro-6H-pyrrolo[3,4-g]quinolin-5-yl)methanesulfonamide (500 g, 0.875 mol). Tetrahydrofuran (1 L) was charged and the resulting slurry was cooled to an internal temperature of −15 to 5° C. under nitrogen atmosphere. To the reaction mixture, 2 M lithium borohydride in THF (481 mL, 0.962 mol, 1.1 equiv) was charged over 1.5 hours. The reaction mixture was heated to reflux, and maintained for 1 hour. After 1 hour of refluxing, methanol (39 mL, 0.962 mol, 1.1 eq.) was added over 1.5 hours. The reaction mixture was maintained at reflux for an additional hour and additional methanol (39 mL, 0.962 mol, 1.1 equiv) was charged over 1.5 hours. The reaction mixture was checked for completion by ¹⁹F NMR (typically 1 hour). When the reaction was judged complete, methanol (117 mL, 3.3 equiv 2.886 mol), was added. The reaction mixture was cooled to 19-25° C. To the reaction mixture, isopropyl acetate (3.5 L) was added. A 5 M aqueous ammonium chloride solution (680 g of ammonium chloride dissolved in 2.5 L of water) was charged. It was agitated for 30 minutes to insure adequate mixing. After agitation, the layers were allowed to separate for 15 minutes.

To the isolated organic layer, brine (625 g sodium chloride dissolved in 2.5 L) was charged. It was agitated for 30 minutes to insure adequate mixing. After agitation, layers were allowed to separate for 15 minutes. The isolated organic layer was then concentrated to 1.5 L. The water content was checked by KF analysis (must be <0.2 wt %). If KF is not ≦0.2 wt %, the solution was charged with more isopropyl acetate and concentrated to 1.5 L. This process was repeated until KF is ≦0.2 wt %. The solution was allowed to cool to 20° C. Once a slurry was achieved, the mixture was charged with 3.5 L of heptane over 2 hours. The reaction mixture was concentrated to 2.5 L and charged with 2.5 L of heptane. The slurry was sampled and the mole percentage of isopropyl acetate was determined. If the mole percentage of isopropyl acetate is above 2%, this-last step is repeated until it is below 2 mol %. The mixture is cooled to 20° C. The slurry is filtered, and the resultant cake is washed with 2 L of heptane. The product was dried in a vacuum oven at 40-50° C. until a stable mass was obtained. The final amount of desired material, N-(7-(4-fluorobenzyl)-8-oxo-9-(triisopropylsilyloxy)-7,8-dihydro-6H-pyrrolo[3,4-g]quinolin-5-yl)methanesulfonamide, compound 8 wherein PG is triisopropylsilyl, obtained was 467.31 g (95.8% of theoretical yield): ¹H NMR (DMSO-d, 400 MHz) δ 9.50 (s, 1 H), 8.93 (d, 1 H, J=2.4 Hz)) 8.62 (d, 1H, J=7.2 Hz) 7.71-7.74 (dd, 1 H, J₁=4 Hz), 7.34-7.46 (m, 2 H), 7.17-7.24 (m, 2 H), 4.70 (s, 2 H), 4.50 (s, 2 H), 3.02 (s, 3 H), 1.43-1.57 (m, 3 H), 0.90-1.25 (m, 18 H); ¹⁹F NMR (DMSO-d, 376 MHz)δ(−116.65)−(−115.83); HPLC AN=96.719%.

### Example 9

### Preparation of N-(7-(4-fluorobenzyl)-9-hydroxy-8-oxo-7,8-dihydro-6H-pyrrolo[3,4-g]quinolin-5-yl)-N-methylmethanesulfonamide

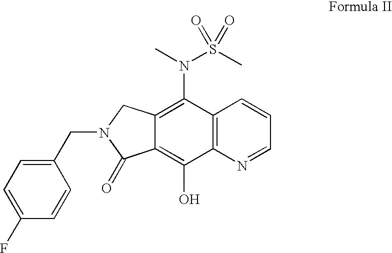

A 5-L reaction flask was charged 390 g (699 mmol) of N-(7-(4-fluorobenzyl)-8-oxo-9-(triisopropylsilyloxy)-7,8-dihydro-6H-pyrrolo[3,4-g]quinolin-5-yl)methanesulfonamide. Acetone (700 mL, 1.0 M, 1.8 vol) was charged and the resulting slurry was cooled to an internal temperature of 15 to 20° C. under a nitrogen atmosphere. An aqueous solution of 45 wt/wt % KOH (65.3 mL, 769 mmol, 1.1 equiv) was charged to the flask at a rate to maintain an internal temperature below 25° C. After a homogeneous solution had formed, the mixture was cooled to an internal temperature of 15 to 20° C., and dimethylsulfate (72.8 mL, 769 mmol, 1.1 equiv) was added at a rate to maintain an internal temperature below 25° C. Upon completion of addition, the reaction mixture was agitated for 40 min without external cooling and a slurry formed. Reaction progress was monitored by HPLC assay. Upon completion of the reaction, MeOH (1.0 L, 2.6 vol) was added to the slurry in one portion, followed by TFA (33.0 mL, 350 mmol, 0.50 equiv), and the slurry was heated to 50° C. Once the reaction was judged completed by HPLC analysis, the slurry was diluted with 800 mL of warm water (42° C.) and the mixture was allowed to cool to room temperature for over a period of 6 h with agitation. After 15 h at room temperature, the slurry was filtered and the solids were rinsed with MeOH (2×1.2 L). The solids were dried under vacuum (<30 inHg) at 50° C. for 3 h. The dried solids (242.6 g, 583 mmol) were then charged to a 5-L reaction flask fitted with a condenser, followed by MeOH (2.8 L, 11.7 vol) and acetonitrile (0.70 L, 3.3 vol). The slurry was heated to reflux and agitated. After 2 h, the slurry was cooled to room temperature slowly and agitated at room temperature for 16 h. The slurry was filtered and the solids were rinsed with MeOH in two portions (750 mL and 500 mL) and then dried under vacuum (<30 “Hg) at 50° C. for 24 h. The desired product, of Formula (II), [228 g, 549 mmol, 78% yield (two steps)] was obtained as a free flowing off-white solid: ¹H NMR (CDCl₃, 300 MHz) δ 8.99 (dd, 1H, J=3.9 and 1.5 Hz), 8.28 (dd, 1H, J=8.7 and 1.5 Hz), 7.65 (dd, 1H, J=8.7 and 1.5 Hz), 7.35 (appt dd, 2H, J=8.4 and 2.4 Hz), 7.06 (appt t, 2H, J=8.4 Hz), 4.96 (d, 1H, J=15.0 Hz), 4.71 (d, 1H, J=17.1 Hz), 4.59 (d, 1H, J=15.0 Hz), 4.38 (d, 1H, J=17.1 Hz), 3.33 (s, 3H), 3.07 (s, 3H); ¹⁹F NMR (CDCl₃, 282 MHz) δ−114.3 (m); MS (ESI) m/z 416 [M+H]⁺.

### Example 10

### Preparation of N-(7-(4-fluorobenzyl)-6,8-dioxo-9-(triisopropylsilyloxy)-7,8-dihydro-6H-pyrrolo[3,4-g]quinolin-5-yl)-N-methylmethanesulfonamide

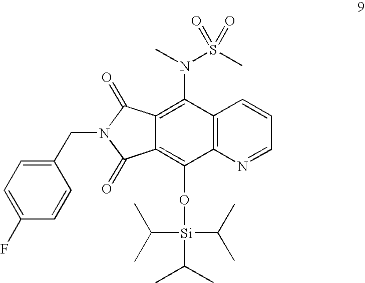 [0160] wherein PG is triisopropylsilyl

50 g (87.45 mmol) of N-(7-(4-fluorobenzyl)-6,8-dioxo-9-(triisopropylsilyloxy)-7,8-dihydro-6H-pyrrolo[3,4-g]quinolin-5-yl)methanesulfonamide (as prepared in Example 7) was dissolved in DMF (150 mL) and cooled to between −10 and 5° C. The reaction vessel was charged with potassium carbonate (325 mesh) (18 g, 131 mmol, 1.5 equiv). A solution of MeI/DMF was prepared by combining methyl iodide (8.2 mL, 131 mmol, 1.5 equiv) with DMF (50 mL) and cooled to between −5 and 5° C. (target 0° C.). The MeI/DMF solution was charged to reaction mixture while maintaining the temperature below 5° C. The progress of the reaction was monitored by HPLC. When the reaction was complete the reaction mixture was warmed to 20° C. The reaction was then charged with isopropyl acetate (1.25 L) and 5 M aqeous ammonium chloride (250 mL) while maintaining the temperature below 30° C. The reaction mixture was then charged with water (150 mL) and mixed for 5 minutes. At completion of mixing, the aqueous layer was separated from the organic layer. The organic layer was charged with water (400 mL) and agitated to mix. At completion of mixing, the aqueous layer was separated from the organic layer. The organic layer was then charged with brine (400 mL) and agitated to mix. At the completion of mixing, the aqueous layer was separated from the organic layer. The organic layer was concentrated to 350 mL by distillation. To this concentrate was charged heptane (1 L) and the resulting slurry was sampled to determine the mole percentage of isopropyl acetate relative to heptane.

If the mole percentage of isopropyl acetate was above 2%, the heptane dilution and concentration steps were repeated until the mole percentage was below 2%. The slurry was cooled to 20° C., and stirred for 1-24 hours. The slurry was then filtered and washed with heptane (2×100 mL) and dried in a vacuum oven at 40° C. yielding 43.69 g. (85.3% of theoretical yield) of N-(7-(4-fluorobenzyl)-6,8-dioxo-9-(triisopropylsilyloxy)-7,8-dihydro-6H-pyrrolo[3,4-g]quinolin-5-yl)-N-methylmethanesulfonamide, compound 9, wherein PG is triisopropylsilyl.

Analytical data: ¹H NMR (DMSO-d, 400 MHz) δ 9.05 (dd, 1 H, J=4.0 and 6.8 Hz), 8.59 (dd, 1 H, J=1.6 and 10.0 Hz), 7.89 (dd, 1 H, J=4.4 and 8.4 Hz) 7.34-7.43 (m, 2 H), 7.09-7.17 (m, 2 H), 4.70-4.76 (m, 2 H), 3.31 (s, 3 H), 3.13 (s, 3 H), 0.79-1.00 (m, 21 H); HPLC AN=98.490%

### Example 11

### Alternate Preparation of N-(7-(4-fluorobenzyl)-9-hydroxy-8-oxo-7,8-dihydro-6H-pyrrolo[3,4-g]quinolin-5-yl)-N-methylmethanesulfonamide

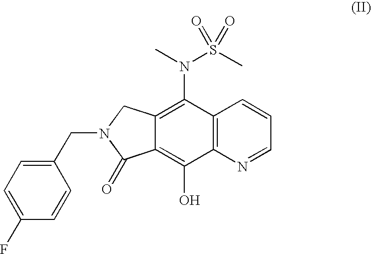

A 20-L reaction flask was charged with 883 g (1.5 mol) of N-(7-(4-fluorobenzyl)-6,8-dioxo-9-(triisopropylsilyloxy)-7,8-dihydro-6H-pyrrolo[3,4-g]quinolin-5-yl)-N-methylmethanesulfonamide followed by THF (3.4 L, 0.44 M, 3.9 vol). The resulting solution was cooled to an internal temperature of 5° C. under a nitrogen atmosphere. A 2.0 M solution of LiBH₄ in THF (1.5 L, 3.0 mol, 2.0 equiv) was charged to the flask at a rate to maintain an internal temperature below 15° C. A brick red solution results. The mixture was cooled to −10° C. and a solution of MeOH in THF (430 mL, 11 mol, 7.0 equiv in 580 mL of THF) was charged to the reaction mixture over a period of 1 h to activate the borohydride. The rate of MeOH addition should be adjusted to maintain an internal temperature below 5° C. The progress of the reaction was monitored by HPLC via the formation of diastereomers of 7-(4-fluorobenzyl)-6-hydroxy-5-(1-(methylsulfonyl)-ethyl)-9-(triisopropylsilyloxy)-6,7-dihydrocyclopenta[g]quinolin-8-one,

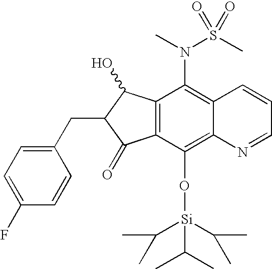

The major (non-polar isomer): Red 0.46 (ethyl acetate/hexanes=1/1); ¹H NMR (CDCl₃, 300 MHz) δ 8.89 (dd, 1H, J=3.9 and 1.5 Hz), 8.46 (dd, 1H, J=8.4 and 1.5 Hz), 7.61 (dd, 1H, J=8.4 and 3.9 Hz), 7.38 (appt dd, 2H, J=8.4 and 5.4 Hz), 7.03 (appt t, 2H, J=8.7 Hz), 5.64 (d, 1H, J=11.1 Hz), 5.14 (d, 1H, J=15.0 Hz), 4.33 (d, 1H, J=15.0 Hz), 3.29 (s, 3H) 3.27 (s, 3H), 3.25 (d, 1H, J=11.1 Hz), 1.56 (m, 3H, 7.4 Hz), 1.15 (d, 18H, J=7.4 Hz); ¹⁹F NMR (CDCl₃, 282 MHz) δ−115.4 (m).); MS (ESI) m/z 586 [M−H]⁻.

The minor (polar isomer): R_{f}=0.26 (ethyl acetate/hexanes=1/1); ¹H NMR (CDCl₃, 300 MHz) δ 8.90 (dd, 1H, J=4.2 and 1.5 Hz), 8.31 (dd, 1H, J=8.4 and 1.5 Hz), 7.61 (dd, 1H, J=8.4 and 4.2 Hz), 7.31 (appt dd, 2H, J=8.4 and 6.0 Hz), 7.01 (appt t, 2H, J=8.6 Hz), 5.97 (d, 1H, J=9.9 Hz), 4.83 (d, 1H, J=15.0 Hz), 4.40 (d, 1H, J=15.0 Hz), 3.40 (s, 3H), 3.16 (d, 1H, J=9.9 Hz), 3.10 (s, 3H), 1.55 (m, 3H, 7.4 Hz), 1.15 (dd, 18H, J=7.4 and 5.5 Hz); ¹⁹F NMR (CDCl₃, 282 MHz) δ −115.3 (m); MS (ESI) m/z 586 [M−H]⁻.

Upon completion of the reaction, an aqueous solution of NH₄Cl (440 g, 8.3 mol, 5.5 equiv in 2.5 L of water) was added to the reaction mixture at 0° C. to quench excess LiBH₄. The reaction mixture was diluted with iPrOAc (17 L), agitated to mix, the layers were allowed to form, and the phases were separated. The organic layer was washed with brine (8 L) and the phases separated. The organic layer was charged to a 20-L reaction flask and concentrated at atmospheric pressure to 5-10% of the original volume. After cooling the mixture to room temperature, the flask was charged with 7 L of DCM and triethylsilane (2.4 L, 15 mol, 10 equiv). 7 L of trifluoroacetic acid (TFA) was added last (˜0.1 M, 17-19 volume including the 2-3 volume of iPrOAc). HPLC was used to monitor the progress of the reaction through the disappearance of both 7-(4-fluorobenzyl)-6-hydroxy-5-(1-(methylsulfonyl)ethyl)-9-(triisopropylsilyloxy)-6,7-dihydrocyclopenta[g]quinolin-8-one diastereomers. Upon completion of the reduction, MeOH (4.0 L, 4.5 vol) was added portion-wise, and the progress of the reaction was monitored by HPLC assay through the disappearance of N-(7-(4-fluorobenzyl)-8-oxo-9-(triisopropylsilyloxy)-7,8-dihydro-6H-pyrrolo[3,4-g]quinolin-5-yl)-N-methylmethanesulfonamide. Upon completion of silyl-deprotection, the organics were concentrated at atmospheric pressure to 5 volumes. The phases were split and the top silyl-layer was discarded. The bottom layer(s) was diluted with 2.3 L of MeOH and 4.0 L of MTBE, and the homogeneous mixture was seeded with N-(7-(4-fluorobenzyl)-9-hydroxy-8-oxo-7,8-dihydro-6H-pyrrolo[3,4-g]quinolin-5-yl)-N-methylmethanesulfonamide (9.4 g, ˜1 wt/wt %). After the slurry developed, an additional 0.8 L of MTBE was added. After 20 h, the slurry was filtered and the solids were washed with MeOH (2×1 L) and MTBE (2×1 L). The solids were dried briefly in the filter with a flow of nitrogen and isolated to afford the desired product, N-(7-(4-fluorobenzyl)-9-hydroxy-8-oxo-7,8-dihydro-6H-pyrrolo[3,4-g]quinolin-5-yl)-N-methylmethanesulfonamide, compound of Formula (II), as a free flowing off-white solid [481 g, 1.16 mol, 77% yield (three steps), 98.3% ES, 98.7% AN]. Analytically pure product can be obtained by performing the following procedure. Reprocessing procedure: A slurry of the compound of (Formula II) in 15 vol of a 1:4 mixture of acetonitrile to MeOH was heated to reflux and maintained for 2 h followed by slow cooling to room temperature. The solids were isolated by filtration, rinsed thoroughly with MeOH (2×1.5 volumes), and dryed under vacuum (<30 inHg) at 50° C. to afford N-(7-(4-fluorobenzyl)-9-hydroxy-8-oxo-7,8-dihydro-6H-pyrrolo[3,4-g]quinolin-5-yl)-N-methylmethanesulfonamide [401 g, 0.97 mol. 89% recovery (451 g input), 100.1% ES, 99.9% AN].

### Example 12

### Preparation of N-(7-(4-fluorobenzyl)-9-hydroxy-8-oxo-7,8-dihydro-6H-pyrrolo[3,4-g]quinolin-5-yl)methanesulfonamide

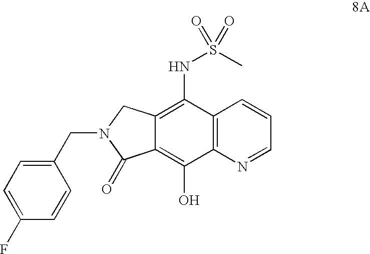

In a reaction vessel was charged with 0.50 g (1.2 mmol) of N-(7-(4-fluorobenzyl)-9-hydroxy-6,8-dioxo-7,8-dihydro-6H-pyrrolo[3,4-g]quinolin-5-yl)methane-sulfonamide followed by THF (4.0 mL, 0.30 M, 8 vol) under an atmosphere of nitrogen. To the slurry was added LiBH₄ (2.0 M solution in THF, 2.4 mL, 4.8 mmol, 4 equiv) dropwise over 5 minutes. After a homogeneous solution had formed, the mixture was warmed to 60° C. and allowed to stir for 16 h. MeOH (3 mL) was added to the reaction mixture followed by TFA (0.60 mL, 0.81 mmol, 0.67 equiv). The mixture was seeded with ˜10 mg of N-(7-(4-fluorobenzyl)-9-hydroxy-8-oxo-7,8-dihydro-6H-pyrrolo[3,4-g]quinolin-5-yl)methanesulfonamide (2.5 mole %) and cooled to rt. The resulting slurry was diluted with MTBE (5 mL) then isolated by filtration. The solids were rinsed with MeOH (2×5 mL) and MTBE (2×5 mL), and dried under vacuum (<30 inHg) at 50° C. to yield the desired product N-(7-(4-fluorobenzyl)-9-hydroxy-8-oxo-7,8-dihydro-6H-pyrrolo[3,4-g]quinolin-5-yl)methanesulfonamide 8A (0.10 g, 0.25 mmol, 21% yield, 93.0% AN); ¹H NMR (d6-DMSO, 400 MHz); 9.71 (s, 1H), 9.04 (d, 1H, J=4.1 Hz), 8.73 (d, 1H, J=8.6 Hz), 7.86 (dd, 2H, J=8.6 and 4.1 Hz), 7.36 (appt t, 2H, J=9.5 Hz), 7.13 (appt t, 2H, J=8.7 Hz), 4.74 (s, 2H), 3.07 (s, 6H); ¹⁹F NMR (d6-DMSO, 376 MHz); (pent, 1F, J=4.6 Hz); MS (ESI) m/z 402 [M+H]⁺.

### Example 13

### Alternate Preparation of N-(7-(4-fluorobenzyl)-9-hydroxy-8-oxo-7,8-dihydro-6H-pyrrolo[3,4-g]quinolin-5-yl)-N-methylmethanesulfonamide

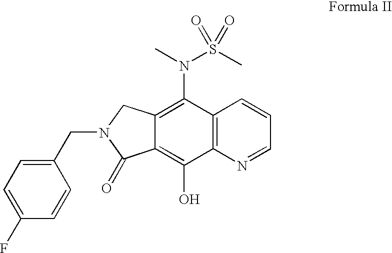

In a reaction vessel was charged 0.050 g (0.13 mmol) of N-(7-(4-fluorobenzyl)-9-hydroxy-8-oxo-7,8-dihydro-6H-pyrrolo[3,4-g]quinolin-5-yl)methanesulfonamide followed by acetone (0.62 mL, 0.20 M, 12 vol) under an atmosphere of nitrogen. KOH (45 wt/wt % solution H₂O, 32 μL, 0.37 mmol, 3 equiv) was added dropwise to the stirred mixture. After a homogeneous solution had formed, dimethylsulfate (35 μL, 0.37 mmol, 3 equiv) was added, and the reaction progress was monitored by HPLC. Upon completion of the reaction, the slurry was diluted with MeOH (1.0 mL). The slurry was filtered and the solids were rinsed with MeOH (2×1 mL) and dried under vacuum(<30 inHg) at 50° C. to afford the desired product, N-(7-(4-fluorobenzyl)-9-hydroxy-8-oxo-7,8-dihydro-6H-pyrrolo[3,4-g]quinolin-5-yl)-N-methylmethanesulfonamide, Formula (II) (25 mg, 0.060 mmol, 48% yield, 95.9% AN).

### Example 14

### Preparation of Salts N-(7-(4-fluorobenzyl)-9-hydroxy-8-oxo-7,8-dihydro-6H-pyrrolo[3,4-g]quinolin-5-yl)-N-methylmethanesulfonamide

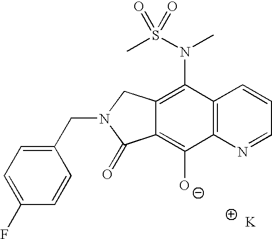

### A. Preparation of the Potassium Salt of N-(7-(4-fluorobenzyl)-9-hydroxy-8-oxo-7,8-dihydro-6H-pyrrolo[3,4-g]quinolin-5-yl)-N-methylmethanesulfonamide

N-(7-(4-fluorobenzyl)-9-hydroxy-8-oxo-7,8-dihydro-6H-pyrrolo[3,4-g]quinolin-5-yl)-N-methylmethanesulfonamide was combined with ethanol to form a slurry. The slurry was agitated and heated to a reflux temperature of 78-79° C. A potassium hydroxide (45% in water) solution in ethanol was charged to the refluxing ethanol slurry. The reaction mixture was agitated and heated till the slurry reached reflux temperature (78-79° C.) where it was maintained for at least one hour. The reaction mixture was slowly cooled over a three hour period to 20 to 22° C. The slurry was filtered and the wet solids were washed with ethanol. The solids were dried under vacuum at 50° C. to afford the potassium salt of N-(7-(4-fluorobenzyl)-9-hydroxy-8-oxo-7,8-dihydro-6H-pyrrolo[3,4-g]quinolin-5-yl)-N-methylmethanesulfonamide (99% yield).

### B. Preparation of Alternate Phenolic Salts

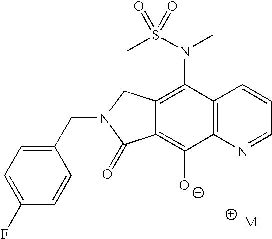

Following the procedure taught in this example, and substituting other base solutions, in combination with a solvent or combination of solvents selected from dimethylformamide, N-methylpyrrolidinone, ethanol, methanol, isopropanol, dimethylacetamide, N-ethylpyrrolidinone, acetone, methyl tert-butyl ether, the following phenolic salts were obtained:
1) Potassium,
2) Sodium,
3) Ethanolamine,
4) Ammonium,
5) Diethylamine,
6) Tromethamine,
7) Benzathine,
8) L-lysine,
9) Ethylene diamine,
10) Deanol,
11) Piperazine,
12) 3-(1H-imidazol-1-yl)-1-propanamine,
13) 1,3-diamino-2-propanol,
14) 2-(benzylamino)ethanol,
15) 4-[2-(4-morpholinyl)ethyl]morpholine,
16) dioctylamine,
17) trans 1,4-diaminocyclohexane, and
18) 1,2-dimethylaminoethane (product to amine/2:1 ratio).

### C. Preparation of Pyridyl Salts

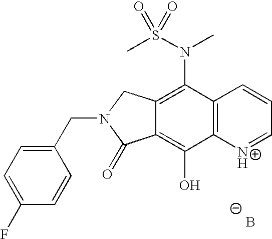

Following the procedure taught in this example, and substituting the base solution with an acid solution, in combination with a solvent or combination of solvents selected from N-methylpyrrolidinone, and ethanol, the following pyridyl salts were obtained:
1) Hydrochloric acid,
2) Methanesulfonic acid,
3) Sulfuric Acid, and
4) Naphthylenesulfonic acid.

All publications, patents, and patent documents are incorporated by reference herein, as though individually incorporated by reference. While the present invention has been described with reference to the specific embodiments thereof, it should be understood that various changes and substitutions may be made by those of ordinary skill in the art without departing from the true spirit and scope of the invention. In addition, many modifications and substitutions may be made to adapt a particular composition of matter, process, process step or steps to the objective, spirit and scope of the present invention. All such modifications are intended to be within the scope of the claims appended hereto.

What is claimed:

1. A method of preparing a compound or a pharmaceutically acceptable salt of Formula (II) 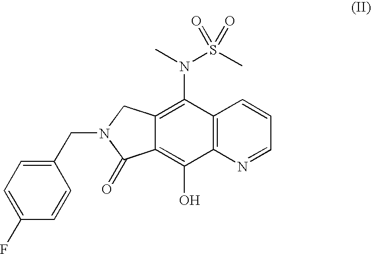
comprising contacting a compound of the Formula, 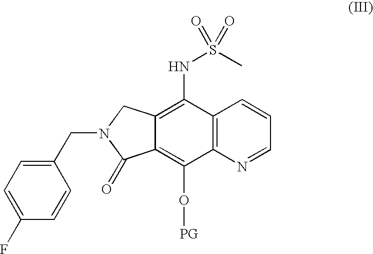
wherein PG is a protecting group with a methylation agent and an acid.

2. The method of claim 1 wherein said methylation agent is followed by an acid.

3. The method of claim 1 wherein said protecting group is a silyl-ether.

4. The method of claim 3 wherein said alkyl substituted silyl ether is tri-isopropylsilyl ether.

5. The method of claim 1 wherein said methylation reagent is a methyl halide.

6. The method of claim 1 wherein said methylation reagent is methyl iodide or dimethylsulfate.

7. The method of claim 1 further comprising contacting a compound of Formula (I), 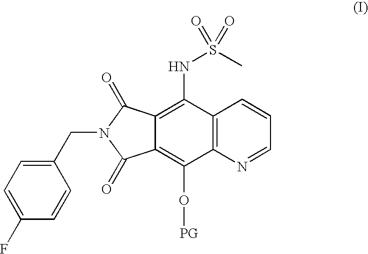
with a reducing agent to provide the compound of said Formula (III),

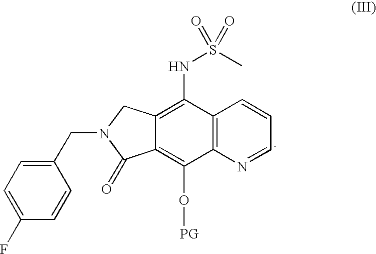

8. The method of claim 9 wherein said reducing agent is LiBH₄.

9. A method of preparing a compound of Formula (II) 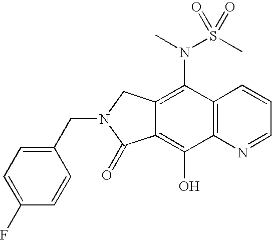
comprising contacting a compound of the Formula, 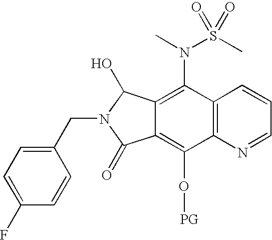
wherein PG is a protecting group with a reducing agent and an acid.

10. The method of claim 9 wherein said protecting group is a silyl-ether.

11. The method of claim 10 wherein said silyl ether is tri-isopropylsilyl ether.

12. The method of claim 9 wherein said reducing agent is LiBH₄.

13. The method of claim 9 further comprising contacting a compound of the Formula, 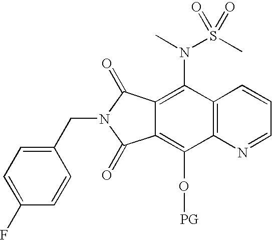
with a reducing agent to provide the compound of said Formula,

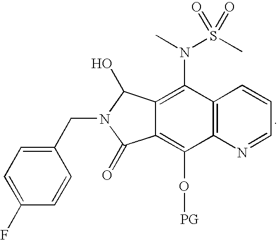

14. The method of claim 13 wherein said reducing agent is LiBH₄.

15. The method of claim 13 further comprising contacting a compound of the Formula 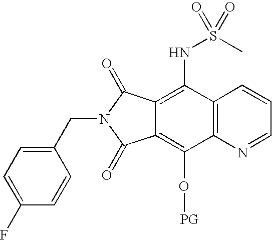
with a methylation reagent to provide the compound of said Formula

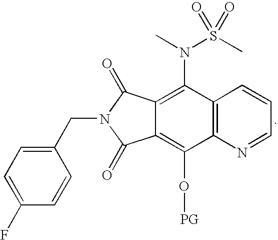

16. The method of claim 15 wherein said methylation reagent is a methyl halide.

17. The method of claim 15 wherein said methylation reagent is methyl iodide or dimethylsulfate.

18. A method of preparing a compound of Formula (II) 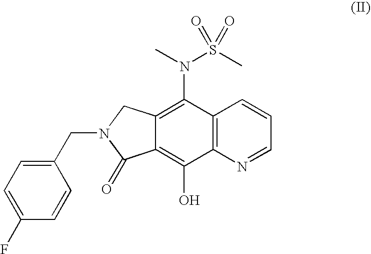
comprising contacting the compound of Formula 8A 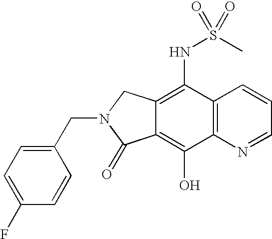
with a methylation reagent.

19. The method of claim 18 wherein said methylation reagent is a methyl halide.

20. The method of claim 18 wherein said methylation reagent is methyl iodide or dimethylsulfate.

21. The method of claim 18 further comprising contacting a compound of Formula IA 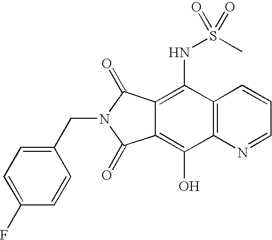
with a reducing agent to provide the a compound of said Formula 8A.

22. The method of claim 21 wherein said reducing agent is LiBH₄.

23. A method of preparing a compound of Formula (II) 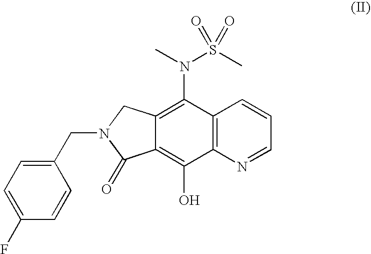
comprising,
a) contacting a compound of Formula IA 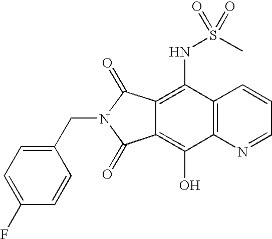
with a reducing agent; and
b) contacting the resultant compound of step a) with a methylation reagent to provide the desired compound.

24. A method of preparing a compound of Formula (II) 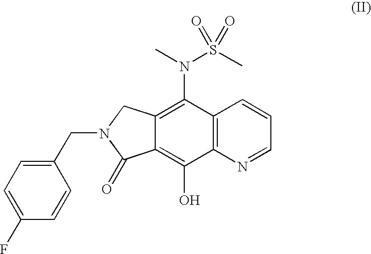
comprising,
a) contacting a compound of the Formula 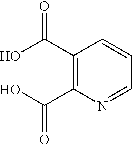
with acetic anhydride to provide a compound of the Formula 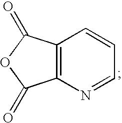
b) contacting said resultant compound of step a) with isopropanol to provide a compound of the Formula 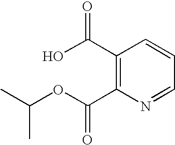
c) contacting said resultant compound of step b) with ammonia and a methylsulfonation reagent to provide a compound of the Formula 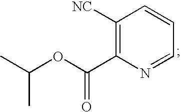
d) contacting said resultant compound of step c) with 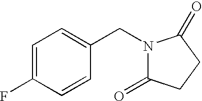
to provide a compound of the Formula 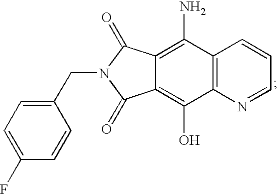
e) contacting said resultant compound of step d) with a protecting agent to provide a compound of the Formula 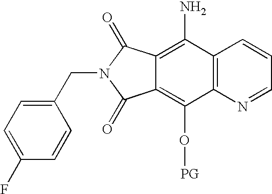
wherein PG is a protecting group;
f) contacting said resultant compound of step e) with a base and methylsulfonation reagent to provide a compound of the Formula 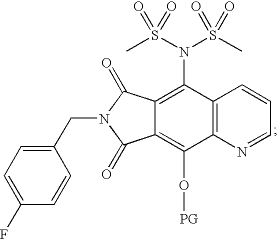
g) contacting said resultant compound of step f) with a base to provide a compound of Formula (I) 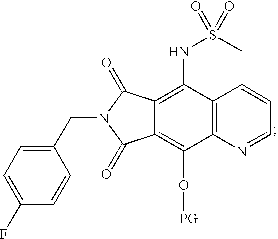
h) contacting said resultant compound of step g) with a methylation reagent to provide a compound of the Formula, 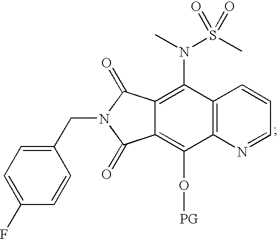
i) contacting said resultant compound of step h) with a first reducing agent to provide a compound of the Formula, 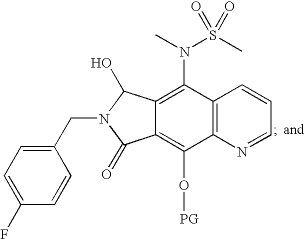
j) contacting said resultant compound of step i) with a second reducing acid and a strong acid to provide the compound of Formula (II).

25. A method of preparing a compound of Formula (II) 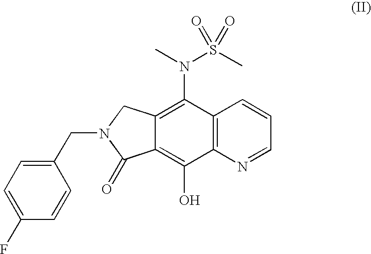
comprising,
a) contacting a compound of the Formula 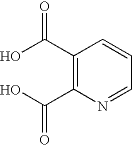
with acetic anhydride to provide a compound of the Formula 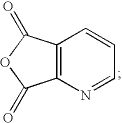
b) contacting said resultant compound of step a) with isopropanol to provide a compound of the Formula 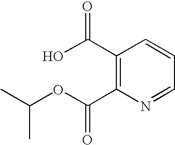
c) contacting said resultant compound of step b) with ammonia and a methylsulfonation reagent to provide a compound of the Formula 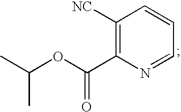
d) contacting said resultant compound of step c) with 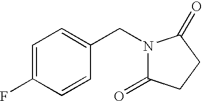
to provide a compound of the Formula 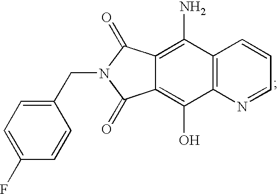
e) contacting said resultant compound of step d) with a protecting agent to provide a compound of the Formula 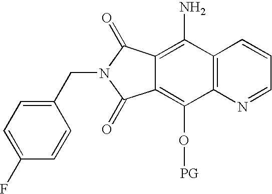
wherein PG is a protecting group;
f) contacting said resultant compound of step e) with a base and methylsulfonation reagent to provide a compound of the Formula 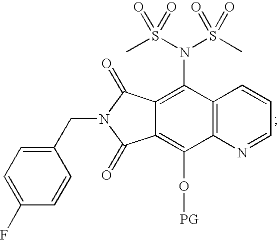
g) contacting said resultant compound of step f) with a base to provide a compound of Formula (I) 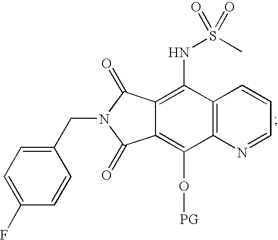
h) contacting said resultant compound of step g) with a reducing agent to provide a compound of the Formula, 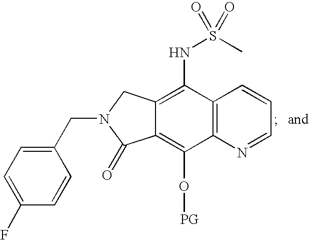
i) contacting said resultant compound of step h) with a methylation agent and an acid to provide a compound of Formula (II).

26. The method of claim 1 further comprising preparing a pharmaceutically acceptable phenolic salt of the compound of Formula (II) 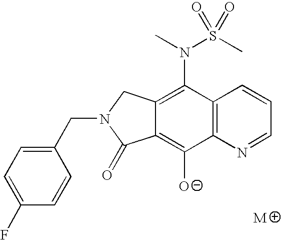
by contacting the compound of Formula (II) 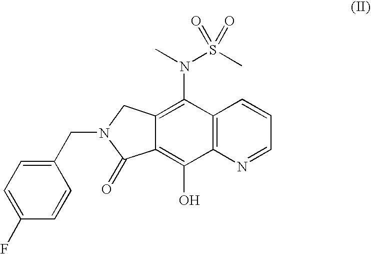
with a base and a solvent or combination of solvents.

27. The method of claim 26 wherein said solvent is selected from the group consisting of dimethylformamide, N-methylpyrrolidinone, ethanol, methanol, isopropanol, dimethylacetamide, N-ethylpyrrolidinone, acetone, and methyl tert-butyl ether or combinations thereof.

28. The method of claim 26 wherein said combination of solvents is selected from the group consisting of dimethylformamide, N-methylpyrrolidinone, ethanol, methanol, isopropanol, dimethylacetamide, N-ethylpyrrolidinone, acetone, methyl tert-butyl ether.

29. The method of claim 26 wherein said base is selected from the group consisting of potassium hydroxide, sodium hydroxide, ethanolamine, ammonium, diethylamine, tromethamine, benzathne, L-lysine, ethylene diamine, deanol, piperazine, 3-(1H-imidazol-1-yl)-1-propanamine, 1,3-diamino-2-propanol, 2-(benzylamino)ethanol, 4-[2-2(4-morpholinyl)ethyl]morphine, dioctylamine, trans 1,4-diaminocyclo-hexane, and 1,2-dimethylaminoethane.

30. The method of claim 26 wherein said base is potassium hydroxide.

31. The method of claim 26 wherein said solvent is ethanol.

32. The method of claim 1 further comprising preparing a pharmaceutically acceptable pyridyl salt the compound of Formula (II) 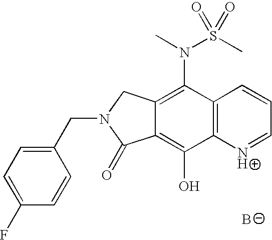
by contacting a compound of Formula (II) 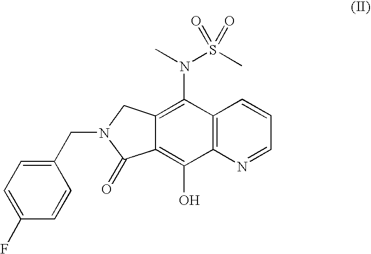
with an acid and a solvent.

33. The method of claim 32 wherein said solvent is selected from the group consisting of N-methylpyrrolidinone and ethanol, or combinations thereof.

34. The method of claim 32 wherein said combination of solvents are N-methylpyrrolidinone and ethanol.

35. The method of claim 32 wherein said acid is selected from the group consisting of hydrochloric acid, methanesulfonic acid, sulfuric acid naphthylenelsulfonic acid, or combinations thereof.

36. A compound of the following Formula: 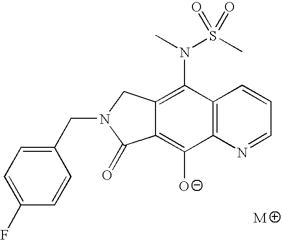
wherein M is sodium or a cation derived from ethanolamine, ammonium, diethylamine, tromethamine, benzathne, L-Iysine, ethylene diamine, deanol, piperazine, 3-(1H-imidazol-1-yl)-1-propanamine, 1,3-diamino-2-propanol, 2-(benzylamino)ethanol, 4-[2-2(4-morpholinyl)ethyl]morphine, dioctylamine, trans 1,4-diaminocyclo-hexane, or 1,2-dimethylaminoethane.

37. A compound of the following Formula: 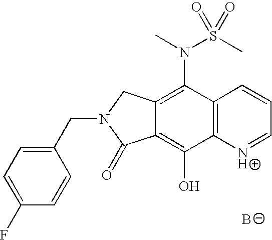
wherein B is chloride, methylsulfonate anion, sulfate, hydrogen sulfate, or naphthylsulfonate anion.

38. A method for preparing a compound of Formula (III), 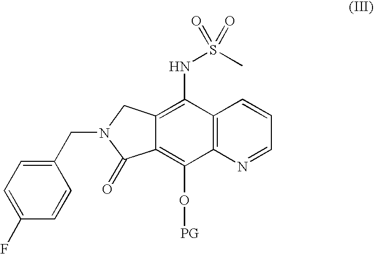
wherein PG is a protecting group, comprising contacting a compound of Formula (I), 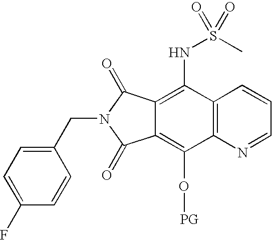
with a reducing agent to provide the compound of Formula (III).

39. The method of claim 38 wherein said reducing agent is LiBH₄.

40. A compound of Formula (III), 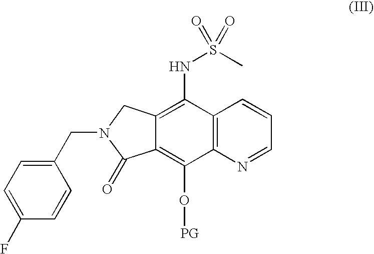
wherein PG is a protecting group; or a salt thereof.

41. The compound of claim 41 wherein PG is tri-isopropylsilyl.
